(19) United States
(12) Patent Application Publication
(10) Pub. No.: US 2022/0105084 A1
(43) Pub. Date: Apr. 7, 2022

(54) PHARMACEUTICAL COMPOSITIONS COMPRISING DEXTROMETHORPHAN AND QUINIDINE FOR THE TREATMENT OF AGITATION IN DEMENTIA

(71) Applicant: Avanir Pharmaceuticals, Inc., Aliso Viejo, CA (US)

(72) Inventors: Joao SIFFERT, San Diego, CA (US); James BERG, San Diego, CA (US)

(21) Appl. No.: 17/347,738

(22) Filed: Jun. 15, 2021

Related U.S. Application Data
(63) Continuation of application No. 16/710,091, filed on Dec. 11, 2019, now abandoned.
Application No. 16/710,091 is a continuation of application No. 16/148,028, filed on Oct. 1, 2018, now abandoned.
Application No. 16/148,028 is a continuation of application No. 15/393,467, filed on Dec. 29, 2016, now abandoned.
Application No. 15/393,467 is a continuation of application No. 14/853,832, filed on Sep. 14, 2015, now abandoned.
Application No. 14/853,832 is a continuation-in-part of application No. 13/750,067, filed on Jan. 25, 2013, now abandoned.
Application No. 13/750,067 is a continuation of application No. 12/820,912, filed on Jun. 22, 2010, now abandoned.
Application No. 12/820,912 is a continuation of application No. 12/181,962, filed on Jul. 29, 2008, now abandoned.
Application No. 12/181,962 is a continuation of application No. PCT/US2007/002931, filed on Feb. 1, 2007.
(60) Provisional application No. 60/854,748, filed on Oct. 27, 2006.
(60) Provisional application No. 60/854,666, filed on Oct. 26, 2006.
(60) Provisional application No. 60/765,250, filed on Feb. 3, 2006.
(60) Provisional application No. 62/050,170, filed on Sep. 14, 2014.
(60) Provisional application No. 62/061,451, filed on Oct. 8, 2014.
(60) Provisional application No. 62/063,122, filed on Oct. 13, 2014.
(60) Provisional application No. 62/063,861, filed on Oct. 14, 2014.
(60) Provisional application No. 62/068,742, filed on Oct. 26, 2014.
(60) Provisional application No. 62/111,053, filed on Feb. 2, 2015.
(60) Provisional application No. 62/111,590, filed on Feb. 3, 2015.
(60) Provisional application No. 62/128,446, filed on Mar. 4, 2015.
(60) Provisional application No. 62/162,140, filed on May 15, 2015.
(60) Provisional application No. 62/165,535, filed on May 22, 2015.
(60) Provisional application No. 62/169,997, filed on Jun. 2, 2015.
(60) Provisional application No. 62/180,026, filed on Jun. 15, 2015.
(60) Provisional application No. 62/193,347, filed on Jul. 16, 2015.
(60) Provisional application No. 62/205,061, filed on Aug. 14, 2015.
(60) Provisional application No. 62/216,636, filed on Sep. 10, 2015.
(60) Provisional application No. 62/217,470, filed on Sep. 11, 2015.

(51) Int. Cl. A61K 31/485; A61K 31/4709; A61K 9/48; A61K 31/49

(52) U.S. Cl. CPC A61K 31/485; A61K 31/49; A61K 9/48; A61K 31/4709

(57) ABSTRACT

This disclosure provides pharmaceutical compositions comprising dextromethorphan in combination with quinidine, and methods for treating agitation and/or aggression in subjects with dementia by administering such compositions.

### PRIORITY

This is a continuation-in-part of U.S. patent application Ser. No. 13/750,067, filed Jan. 25, 2013, which is a continuation of U.S. patent application Ser. No. 12/820,912, filed Jun. 22, 2010, which is a continuation of U.S. patent application Ser. No. 12/181,962, filed Jul. 29, 2008, which is a national stage filing under 35 U.S.C. § 371 of International Application No. PCT/US2007/002931, filed Feb. 1, 2007, which claims priority to U.S. Provisional Application Nos. 60/854,748, filed Oct. 27, 2006; 60/854,666, filed Oct. 26, 2006; and 60/765,250 filed Feb. 3, 2006. In addition, the instant application also claims priority to U.S. Provisional Application Nos. 62/050,170, filed Sep. 14, 2014; 62/061,451, filed Oct. 8, 2014; 62/063,122, filed Oct. 13, 2014; 62/063,861, filed Oct. 14, 2014; 62/068,742, filed Oct. 26, 2014; 62/111,053, filed Feb. 2, 2015; 62/111,590, filed Feb. 3, 2015; 62/128,446, filed Mar. 4, 2015; 62/162,140, filed May 15, 2015; 62/165,535, filed May 22, 2015; 62/169,997, filed Jun. 2, 2015; 62/180,026, filed Jun. 15, 2015; 62/193,347, filed Jul. 16, 2015; and 62/205,061, filed Aug. 14, 2015, 62/216,636, filed Sep. 10, 2015, and 62/217,470, filed Sep. 11, 2015. All of these references are incorporated herein by reference.

### FIELD

This disclosure provides pharmaceutical compositions comprising dextromethorphan in combination with quinidine, and methods for treating agitation and/or aggression and/or associated symptoms in subjects with dementia, such as Alzheimer's disease, by administering such compositions.

### BACKGROUND

Alzheimer's disease is a progressive neurodegenerative disease that eventually leads to death. An estimated 5.4 million Americans have Alzheimer's disease. That number has doubled since 1980 and is expected to be as high as 16 million by 2050 (Brookmeyer et al., Alzheimers Dement. 2011; 7(1):61-73). Among US adults over age 65, prevalence estimates of dementia range from 5% to 15%, with Alzheimer's disease being the most common type of dementia (Kaplan and Sadock's Synopsis of Psychiatry: Behavioral Sciences, 1998; Evans et al., JAMA. 1989; 262(18):2551-6; Losonczy et al., Public Health Reports., 1998; 113:273-80).

Agitation is widely recognized as a common and important clinical feature of Alzheimer's disease and other forms of dementia (Ballard et al., Nat. Rev. Neurol. 2009; 5(5):245-55). Although readily recognized by clinicians and caregivers, a consensus definition of agitation in dementia was only recently developed by the International Psychogeriatric Association (IPA) Agitation Definition Working Group (ADWG) with the following criteria: “1) occurring in patients with a cognitive impairment or dementia syndrome; 2) exhibiting behavior consistent with emotional distress; 3) manifesting excessive motor activity, verbal or physical aggression; and 4) evidencing behaviors that cause excess disability impairing relationships and/or daily activities and are not solely attributable to another disorder (psychiatric, medical, or substance-related)” (Cummings et al., Int. Psychogeriatr. 2015; 27(1)7-17). Agitation and/or aggression are estimated to affect up to approximately 80% of patients with dementia (Ryu et al., Am. J. Geriatr. Psychiatry. 2005; 13(11):976-83; Tractenberg et al., J. Geriatr. Psychiatry. Neurol. 2003; 16(2):94-99) with an increase in prevalence as the disease progresses.

Agitation in patients with dementia is associated with increased functional disability (Rabins et al., Alzheimer's Dement. 2013; 9(2)204-207), worse quality of life (Gonzalez-Salvador et al., Int. J. Geriatr. Psychiatry. 2000; 15(2):181-189), earlier institution (Steele et al., Am. J. Psychiatry. 1990; 147(8):1049-51), increased career burden (Rabins et al., Alzheimer's Dement. 2013; 9(2)204-207, increased healthcare costs (Murman et al., Neurology. 2002; 59(11):1721-29), shorter time to severe dementia (Peters et al., Am. J. Geriatr. Psychiatry. 2014; 22(3):S65-S66), and accelerated mortality (Peters et al., Am. J. Geriatr. Psychiatry. 2014; 22(3):S65-S66). For these reasons, agitation and aggression are the neuropsychiatric symptoms most likely to require pharmacological intervention in Alzheimer's patients (Ballard et al., Nat. Rev. Neurol. 2009; 5(5):245-55). However, there are currently no FDA-approved pharmacological treatments for agitation in Alzheimer's disease, and clinicians ultimately resort to off-label use of antipsychotics, sedatives/hypnotics, anxiolytics, and antidepressants in an attempt to control symptoms (Maher et al., JAMA. 2011; 306(12):1359-69). Unfortunately, these treatments have limited utility given a modest efficacy that is offset by relatively poor adherence, safety, and tolerability (Ballard et al., Nat. Rev. Neurol. 2009; 5(5):245-55; Schneider et al., N. Engl. J. Med. 2006; 355(15):1525-38; Huybrechhts et al., BMJ. 2012; 344:e977). Thus a critical need exists to develop a safe and effective pharmacological intervention for the treatment of agitation in dementia. Such a treatment could profoundly impact patient care, reduce caregiver burden, and potentially improve overall disease prognosis.

### SUMMARY

As described above, there remains an urgent need for additional or improved forms of treatment for agitation, aggression, and/or associated symptoms in dementia, such as Alzheimer's disease. This disclosure provides a method of treating agitation and/or aggression and/or associated symptoms in subjects with dementia, such as Alzheimer's disease, without an increased risk of serious adverse effects.

The present disclosure provides a method for treating agitation and/or aggression and/or associated symptoms in subjects with dementia by administering dextromethorphan in combination with quinidine to a subject in need thereof. The disclosure also encompasses the use of pharmaceutically acceptable salts of either or both dextromethorphan and quinidine in the described methods. In one embodiment the dementia is Alzheimer's type dementia.

In some embodiments, dextromethorphan is administered in an amount ranging from about 10 mg per day to about 200 mg per day, and quinidine is administered in an amount ranging from about 0.05 mg per day to less than about 50 mg per day.

In one embodiment, quinidine is administered in an amount ranging from about 4.75 mg per day to about 20 mg per day.

In another embodiment, dextromethorphan is administered in an amount ranging from about 15 mg per day to about 90 mg per day. In another embodiment, dextromethorphan is administered in an amount ranging from about 20 mg per day to about 45 mg per day.

In some embodiments, either or both of quinidine and dextromethorphan are in the form of a pharmaceutically acceptable salt. In some embodiments, the pharmaceutically acceptable salts include alkalai metals, salts of lithium, salts of sodium, salts of potassium, salts of alkaline earth metals, salts of calcium, salts of magnesium, salts of lysine, salts of N,N′dibenzylethylenediamine, salts of chloroprocaine, salts of choline, salts of diethanolamine, salts of ethylenediamine, salts of meglumine, salts of procaine, salts of tris, salts of free acids, salts of free bases, inorganic salts, salts of sulfate, salts of hydrochloride, and salts of hydrobromide. In some embodiments, dextromethorphan is in the form of dextromethorphan hydrobromide. In some embodiments, quinidine is in the form of quinidine sulfate.

In some embodiments, dextromethorphan and quinidine are administered in a unit dosage form. In some embodiments, the unit dosage form comprises about 4.75, 9, or 10 mg of quinidine (for example, quinidine sulfate) and about 15 mg, 20 mg, 23 mg, 30 mg, or 45 mg of dextromethorphan (for example, dextromethorphan hydrobromide). In one embodiment, the unit dosage form comprises about 10 mg of quinidine (for example, quinidine sulfate) and about 20 mg, 30 mg, or 45 mg of dextromethorphan (for example, dextromethorphan hydrobromide). In another embodiment, the unit dosage form comprises about 9 mg of quinidine (for example, quinidine sulfate) and about 15 mg or 23 mg of dextromethorphan (for example, dextromethorphan hydrobromide).

In some embodiments, the unit dosage form of dextromethorphan in in the form of a tablet or a capsule.

In some embodiments, the weight ratio of dextromethorphan to quinidine is about 1:1 or less. In some embodiments, dextromethorphan and quinidine are administered in a combined dose in a weight ratio of dextromethorphan to quinidine of 1:1 or less. The weight ratios of dextromethorphan to quinidine can be, for example, about 1:0.68, about 1:0.6, about 1:0.56, about 1:0.5, about 1:0.44, about 1:0.39, about 1:0.38, about 1:0.33, about 1:0.25, and about 1:0.22.

In one embodiment, dextromethorphan and quinidine are administered as one combined dose per day.

In one embodiment, dextromethorphan and quinidine are administered as at least two combined doses per day.

In some embodiments, the improvement by treatment with dextromethorphan in combination with quinidine in agitation and/or aggression and/or associated symptoms in subjects with dementia, such as Alzheimer's disease, may be measured by improvements of one or more of the following scores:
Neuropsychiatric Inventory (NPI) agitation/aggression domain;
NPI total;
Composite of NPI agitation/aggression, irritability/lability, aberrant motor behavior, and anxiety domains (NPI4A);
Composite of NPI agitation/aggression, irritability/lability, aberrant motor behavior, and disinhibition domains (NPI4D);
NPI caregiver distress—agitation/aggression domain;
Modified Alzheimer Disease Cooperative Study—Clinical Global Impression of Change (ADCS-CGIC) score of agitation; and/or
Patient Global Impression of Change (PGI-C) score of agitation.

In one embodiment, the subject's NPI score for agitation/aggression is reduced by at least 1.5 compared to untreated subjects or subjects administered a placebo.

In one embodiment, the subject's NPI4A score is reduced by at least 2.4 compared to untreated subjects or subjects administered a placebo.

In one embodiment, the subject's NPI4D score is reduced by at least 3.0 compared to untreated subjects or subjects administered a placebo.

In one embodiment, the subject's ADCS-CGIC score of agitation is improved by at least 0.5 compared to untreated subjects or subjects administered a placebo.

In one embodiment, the subject's PGI-C score of agitation is improved by at least 0.6 compared to untreated subjects or subjects administered a placebo.

The pharmaceutical preparations disclosed herein may, optionally, include pharmaceutically acceptable carriers, adjuvants, fillers, or other pharmaceutical compositions, and may be administered in any of the numerous forms or routes known in the art.

The methods disclosed herein may also optionally include administration of dextromethorphan and quinidine in conjunction with other therapeutic agents, such as, for example, one or more therapeutic agents known or identified for treatment of Alzheimer's disease.

It is to be understood that the foregoing general description and the following detailed description are exemplary and explanatory only, and are intended to provide further, non-limiting explanation of the disclosure.

### BRIEF DESCRIPTION OF DRAWINGS

FIG. 1 provides the study design for the Agitation in Alzheimer's Disease Clinical Study. “Dextromethorphan/quinidine 20/10” refers to a dose of 20 mg dextromethorphan and 10 mg quinidine. QD and BID refer to dosages of once daily and twice per day, respectively. Asterisk (*) denotes participants who discontinued prior to the Week 1 visit and therefore did not have any post-baseline data for the primary efficacy endpoint.

FIG. 2 provides a schematic of the Consolidated Standards of Reporting Trials (CONSORT) patient flow chart for the Agitation in Alzheimer's Disease Clinical Study described herein. The populations denoted with * represent those included in the sequential parallel comparison design (SPCD).

FIG. 3 illustrates the mean NPI agitation/aggression scores in stage 1 for subjects included in the Agitation in Alzheimer's Disease Clinical Study described herein, which utilized the sequential parallel comparison design (or SPCD). P-values, calculated from an Analysis of Covariance (ANCOVA) model with treatment as fixed effect and baseline as covariate, are given for each visit. ^{a}=Observed cases.

FIG. 4 illustrates the mean NPI agitation/aggression scores in stage 2 for subjects included in the Agitation in Alzheimer's Disease Clinical Study (utilizing the SPCD). P-values, calculated from an ANCOVA model with treatment as fixed effect and baseline as covariate, are given for each visit. ^{a}=Observed cases.

FIG. 5 illustrates the mean NPI agitation/aggression scores in the 10-week secondary analysis of the Agitation in Alzheimer's Disease Clinical Study described herein. The 10-week secondary analysis included only subjects who remained in the same treatment assignment during the study, i.e., were randomized to receive only dextromethorphan/quinidine or only placebo for the entirety of the study, thus simulating a parallel design. P-values, calculated from ANCOVA model with treatment as fixed effect and baseline as covariate, are given for each visit. ^{a}=Observed cases.

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed as limiting.

All references cited herein, including, but not limited to, published and unpublished applications, patents, and literature references, are incorporated herein by reference in their entirety and are hereby made a part of this specification.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

### Definitions

The term “comprising” as used herein is synonymous with “including,” “containing,” or “characterized by,” and is inclusive or open-ended and does not exclude additional, unrecited elements or method steps.

The terms “ameliorate” and “treat” are used interchangeably and include therapeutic. Both terms mean improve, decrease, suppress, attenuate, diminish, arrest, or stabilize the development or progression of a disease (e.g., a disease or disorder delineated herein) or symptoms of a disease, alone or in constellations (e.g. syndrome).

The term “treat” is used herein to mean to relieve or alleviate at least one symptom of a disease in a subject. For example, in relation to behavioral disorders, the term “treat” may mean to relieve or alleviate agitation and/or aggression and any combination of its manifestations (e.g. pacing, rocking, gesturing, pointing fingers, restlessness, performing repetitious mannerisms, yelling, speaking in an excessively loud voice, using profanity, screaming, shouting, grabbing, shoving, pushing, resisting, hitting others, kicking objects or people, scratching, biting, throwing objects, hitting self, slamming doors, tearing things, destroying property, etc.) and associated behaviors (e.g. irritability, lability, aberrant motor behavior, anxiety, and disinhibition). Within the meaning of the present disclosure, the term “treat” also denotes to arrest, delay the onset (i.e., the period prior to clinical manifestation of a disease) and/or reduce the risk of developing or worsening a disease.

“Disease” means any condition or disorder that damages or interferes with the normal function of a cell, tissue, organ or an organism.

The term “dementia” refers to a general mental deterioration due to organic or psychological factors; characterized by disorientation, impaired memory, judgment, and intellect, and a shallow labile affect. Dementia herein includes vascular dementia, ischemic vascular dementia, frontotemporal dementia, Lewy body dementia, Alzheimer's dementia, etc. The most common form of dementia is associated with Alzheimer's disease.

“Alzheimer's disease” refers to progressive mental deterioration manifested by memory loss, confusion, and disorientation, generally beginning later in life, and commonly resulting in death in 5-10 years. Alzheimer's disease can be diagnosed by a skilled neurologist or clinician. In one embodiment, the subject with AD will meet National Institute of Neurological and Communicative Disorders and Stroke/Alzheimer's Disease and Related Disorders Association (NINCDS/ADRDA) criteria for the presence of probable AD.

The term “agitation,” as used in this disclosure, is includes the definition of agitation as described by Cummings et al., International Psychogeriatrics. 2015; 27(1):7-17. Broadly, Cummings et al. define agitation as: 1) occurring in patients with a cognitive impairment or dementia syndrome; 2) exhibiting behavior consistent with emotional distress (e.g. rapid changes in mood, irritability, outbursts, etc.) and the behavior has been persistent or frequently recurrent for a minimum of two weeks and is a change from the patient's usual behavior; 3) the behaviors are severe enough to produce excess disability; and 4) and the agitation is not solely attributable to another disorder (psychiatric, suboptimal care conditions, medical, or substance-related). Cummings et al. define behaviors consistent with emotional distress as “(a) [e]xcessive motor activity ([e.g.] pacing rocking, gesturing, pointing fingers, restlessness, performing repetitious mannerisms)[;] (b) [v]erbal aggression (e.g. yelling, speaking in an excessively loud voice, using profanity, screaming, shouting)[;] [and] (c)[p]hysical aggression (e.g. grabbing, shoving, pushing, resisting, hitting others, kicking objects or people, scratching, biting, throwing objects, hitting self, slamming doors, tearing things, and destroying property)” (Cummings et al., International Psychogeriatrics. 2015; 27(01); 7-17). In Cummings' definition, excess disability due to severity of behavior is in the clinician's opinion beyond what is due to cognitive impairment and include significant impairment in at least one of the following: (a) interpersonal relationships, other aspects of social functioning, or ability to perform or participate in daily living activities (Cummings et al., International Psychogeriatrics. 2015; 27(01); 7-17). The definition of “agitation”, when used alone, also includes the term “aggression.”

The term “associated symptoms” as used herein refers to symptoms associated with a patient that meets criteria for a cognitive impairment or dementia syndrome (e.g. Alzheimer's disease, frontotemporal dementia, Lewy body dementia, vascular dementia, other dementias, a pre-dementia cognitive impairment syndrome such as mild cognitive impairment or other cognitive disorder). Associated symptoms include, for example, behaviors that are associated with observed or inferred evidence of emotional distress (e.g. rapid changes in mood, irritability, outbursts). In some instances, the behavior is persistent or frequently recurrent for a minimum of two weeks' and represents a change from the patient's usual behavior. The term “associated symptoms” also includes excessive motor activity (examples include: pacing, rocking, gesturing, pointing fingers, restlessness, performing repetitious mannerisms), verbal aggression (e.g. yelling, speaking in an excessively loud voice, using profanity, screaming, shouting), physical aggression (e.g. grabbing, shoving, pushing, resisting, hitting others, kicking objects or people, scratching, biting, throwing objects, hitting self, slamming doors, tearing things, and destroying property).

The term “combination” applied to active ingredients is used herein to define a single pharmaceutical composition (formulation) comprising both drugs of the disclosure (e.g., dextromethorphan and quinidine) or two separate pharmaceutical compositions (formulations), each comprising a single drug of the disclosure (e.g., dextromethorphan or quinidine), to be administered conjointly.

Within the meaning of the present disclosure, the term “conjoint administration” is used to refer to administration of dextromethorphan and quinidine simultaneously in one composition, or simultaneously in different compositions, or sequentially. For sequential administration to be considered “conjoint,” the dextromethorphan and quinidine are administered separated by a time interval sufficient to permit the resultant beneficial effect for treating, preventing, arresting, delaying the onset of and/or reducing the risk of developing a behavioral disorder associated with a central nervous system (CNS) disorder in a subject. For example, the dextromethorphan and quinidine may be administered on the same day (e.g., each once or twice daily).

The term “therapeutically effective” applied to dose or amount refers to that quantity of a compound or pharmaceutical composition that is sufficient to result in a desired activity upon administration to a subject in need thereof. As used herein with respect to the pharmaceutical compositions comprising dextromethorphan, the term “therapeutically effective amount/dose” is used interchangeably with the term “neurologically effective amount/dose” and refers to the amount/dose of a compound or pharmaceutical composition that is sufficient to produce an effective neurological response, i.e., improvement of a behavioral disorder associated with a CNS disorder, upon administration to a subject.

The phrase “pharmaceutically acceptable,” as used in connection with compositions of the disclosure, refers to molecular entities and other ingredients of such compositions that are physiologically tolerable and do not typically produce untoward reactions when administered to a subject (e.g., human). In certain embodiments, as used herein, the term “pharmaceutically acceptable” means approved by a regulatory agency of a Federal or a state government or listed in the U.S. Pharmacopeia or other generally recognized pharmacopeia for use in mammals (e.g., humans).

The term “carrier” applied to pharmaceutical compositions of the disclosure refers to a diluent, excipient, or vehicle with which an active compound (e.g., dextromethorphan) is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like. Suitable pharmaceutical carriers are described in “Remington's Pharmaceutical Sciences” by E. W. Martin, 18th Edition.

The term “subject” as used herein includes a mammal (e.g., rodent such as mouse or rat). In some embodiments, the term refers to humans presenting with a behavioral disorder associated with a CNS disorder, such as, agitation, aggression, and/or associated symptoms. The term “subject” also includes a humans presenting with neuropsychiatric symptoms or behavioral symptoms of dementia.

The term “compound,” as used herein, is also intended to include any salts, solvates, or hydrates thereof. Thus, the terms “dextromethorphan” and “quinidine” will be used for ease of use in this application, and will include salt forms thereof.

A salt of a compound of this disclosure is formed between an acid and a basic group of the compound, such as an amino functional group, or a base and an acidic group of the compound, such as a carboxyl functional group. According to another embodiment, the compound is a pharmaceutically acceptable acid addition salt.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, p-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

Unless otherwise specified, the doses described herein refer to the hydrobromide and sulfate salt forms of dextromethorphan and quinidine, respectively. Based on such information, those skilled in the art can calculate corresponding dosages for the respective free-acid or free-base forms of the active ingredient. For example, a dose of 30 mg dextromethorphan hydrobromide (of molecular formula C₁₈H₂₅NO.HBr.H₂O) and 10 mg quinidine sulfate (of molecular formula (C₂₀H₂₄N₂O₂)₂.H₂SO₄.2H₂O) may be administered (corresponding to approximately 22 mg dextromethorphan and 8.3 mg quinidine). Other dosages include, for example, 45 mg dextromethorphan hydrobromide and 10 quinidine sulfate (corresponding to approximately 33 mg dextromethorphan and approximately 8.3 mg quinidine); 15 mg dextromethorphan hydrobromide and 9 mg quinidine sulfate (corresponding to approximately 11 mg dextromethorphan and approximately 7.5 mg quinidine); 20 mg dextromethorphan hydrobromide and 10 mg quinidine sulfate (corresponding to approximately 14.7 mg dextromethorphan and 8.3 mg quinidine); and 23 mg dextromethorphan hydrobromide and 9 mg quinidine sulfate (corresponding to approximately 16.9 mg dextromethorphan and 7.5 mg quinidine).

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

Alzheimer's Disease

Agitation and aggression are highly prevalent in patients with Alzheimer's disease (Tractenberg et al., J. Geriatr. Psychiatry Neurol. 2003; 16(2):94-9; Ryu et al., Am. J. Geriatr. Psychiatry. 2005; 13(11):976-83) and are associated with distress for patients and caregivers, greater risk for institutionalization, and accelerated progression to severe dementia and death (Gilley et al., Psychol. Med. 2004; 34(6):1129-1135; Rabins et al., Alzheimers Dement. 2013; 9(2):2014-7; Salzman et al., J. Clin. Psychiatry. 2008; 69(6):889-898). Although behavioral disturbances are more frequent as the disease progresses, Alzheimer's disease patients can manifest depression, disruptive behaviors (e.g., agitation, aggression) and psychosis at any stage of the disease (Jost and Grossberg, J. Am. Geriatr. Soc. 1996; 44(9):10789-81). This suggests that while some psychiatric symptoms are associated with the progressive nature of the disease, others result from specific phenotypes associated with increased vulnerability in specific brain areas. Frontal cortical circuits are particularly important in terms of aggression, psychosis, and agitation (Jeste et al., Am. J. Psychiat. 1992; 149(2):184-9; Kotria et al., Am. J. Psychiat. 1995; 152(10):1470-5; Lopez et al., J. Neuropsych. Clin. N. 2001; 13(1):50-5; Sultzer et al., J. Neuropsych. Clin. N. 1997; 7:476-84).

A large cross-sectional study examined relationships among the constellation of psychiatric syndromes as a function of disease severity in 1155 patients with probable Alzheimer's disease (Lopez, J. Neuropsych. Clin. N. 2003; 15(3):346-53). Neuropsychiatric symptoms such as anxiety, wandering, irritability, inappropriate behavior, uncooperativeness, and emotional lability were found to be associated with agitation, aggression, and psychosis, which varied according to the severity of the disease, suggesting a progressive deterioration of fronto-temporal limbic structures. Aggression was associated with agitation, uncooperativeness, and emotional lability in mild/moderate stages, and psychosis, uncooperativeness, and irritability in moderate/severe stages. As with aggression, agitation was also associated with frontal lobe symptoms in all stages of the disease, although this was more evident in mild/moderate stages (Lopez, J. Neuropsych. Clin. N. 2003; 15(3):346-53).

Agitation is generally characterized by motor restlessness, a heightened response to stimuli, irritability, and inappropriate and often purposeless motor or verbal activity. Symptoms generally fluctuate over time, occasionally rapidly and are often associated with sleep disturbances (Sachdev and Kruk, Psychiatry. 1996; 30:38-53). Different attempts have been made to further classify subtypes of agitation. Cohen-Mansfield (Cohen-Mansfield, JAGS. 1986; 34:722-7) distinguishes between the presence of an aggressive physical component (e.g., destroying objects, grabbing, fighting), and aggressive verbal component (e.g., screaming, cussing); and a non-aggressive physical component (e.g., pacing), and a non-aggressive verbal component (e.g., continuous questioning).

Nonpharmacologic interventions are recommended as first line therapy for treating agitation and/or aggression, but many patients fail to respond and pharmacotherapy is often needed (Salzman et al., J. Clin. Psychiatry. 2008; 69(6):889-98; Kales et al., J. Am. Geriatr. Soc. 2014; 62(4):762-9; Gitlin et al., JAMA. 2012; 308(19):2020-9). Although many classes of psychotropic drugs are prescribed for agitation, safety concerns and modest or unproven efficacy limit their utility. Antipsychotics have shown benefit for Alzheimer's disease-related psychosis but their use is associated with excess mortality, cerebrovascular events, sedation, falls, cognitive impairment, metabolic syndrome, Parkinsonism, and tardive dyskinesia (Salzman et al., J. Clin. Psychiatry. 2008; 69(6):889-98; Schneider et al., Am. J. Geriatr. Psychiatry. 2006; 14(3):191-210). A recent trial showed that citalopram, a selective serotonin reuptake inhibitor, was associated with improvement in agitation in Alzheimer's disease but was associated with prolonged QTc interval and mild cognitive decline (Porsteinsson et al., JAMA. 2014; 311(7):682-91).

Accumulating clinical evidence suggests that NMDA antagonists may have an effect in controlling agitation in subjects with Alzheimer's disease. Memantine, which is approved for the treatment of Alzheimer's disease, also acts as a non-competitive, low potency NMDA receptor antagonist and inhibits prolonged cell influx of calcium ions (Rogawski and Went, NS Drug Reviews. 2003; 9(3):275-308; Lipton, Current Alzheimer Res. 2005; 2:155-65). A meta-analysis of data from the memantine efficacy trials was conducted to further examine the outcomes in subjects with Alzheimer's disease who had agitation, aggression, or psychosis before entering the trials. Across the studies, improvement in the NPI behavioral symptom cluster was significantly better with memantine than with placebo at 3 and 6 months. Additionally, the incidence of discontinuations due to agitation was 3-fold higher in placebo-treated subjects than in subjects receiving memantine (Wilcock et al., J. Clin. Psychiatry. 2008; 69(3):341-8). A randomized, placebo controlled 12-week study assessed the potential effect of memantine in 153 nursing home subjects with Alzheimer's disease and agitation (Fox et al., Annual Scientific Meeting on the American-Geriatrics Society. 2011; 59:S65-S66). Whereas the primary endpoint, change in the Cohen-Mansfield Agitation Inventory (CMAI), failed to show a statistically significant difference compared to placebo, there were potential benefits suggested by improvements seen in the NPI (p=0.01) and AD-ADL (p=0.04). The severe impairment battery (SIB) also showed a cognitive effect favoring memantine (p=0.02). Another study conducted in community dwelling subjects with moderate to severe Alzheimer's disease receiving donepezil for at least 3 months (N=295) assessed the effects of various permutations of study medication-placebo, as follows: to continue donepezil, discontinue donepezil, discontinue donepezil and start memantine, or continue donepezil and start memantine. Patients received the study treatment for 52 weeks. The patients who received memantine, as compared with those who received placebo-memantine, had scores on the NPI that were lower (indicating fewer behavioral and psychological symptoms) by an average of 4.0 points (99% Cl, 0.6 to 7.4; p=0.002). In contrast, donepezil did not have an effect on NPI scores) (Howard et al., NEJM. 2012; 366:893-903).

As used herein, the total NPI score is the composite of the scores for the standard 12 NPI domains. The NPI is a validated clinical instrument for evaluating psychopathology in a variety of disease settings, including dementia. The NPI is a retrospective caregiver-informant interview covering 12 neuropsychiatric symptom domains: delusions, hallucinations, agitation/aggression, dysphoria/depression, anxiety, euphoria/elation, apathy/indifference, disinhibition, irritability/lability, aberrant motor behaviors, nighttime behavioral disturbances, and appetite/eating disturbances. The scripted NPI interview includes a compound screening question for each symptom domain, followed by a list of interrogatives about domain-specific behaviors that is administered when a positive response to a screening question is elicited. Neuropsychiatric manifestations within a domain are collectively rated by the caregiver in terms of both frequency (0 to 4) and severity (1 to 3), yielding a composite (frequency×severity) symptom domain score of 1 to 12 for each positively endorsed domain. Frequency and severity rating scales have defined anchor points to enhance the reliability of caregiver responses. Caregiver distress is rated for each positive neuropsychiatric symptom domain on a scale anchored by scores of 0 (not distressing at all) to 5 (extremely distressing). As used herein, the NPI4A score is the composite score comprising the NPI agitation/aggression, aberrant motor behavior, irritability/lability, and anxiety domains. As used herein, the NPI4D score is the composite score comprising the NPI agitation/aggression, aberrant motor behavior, irritability/lability, and disinhibition domains.

Additional evidence suggesting glutamate modulation as a potential therapeutic approach for the management of agitation and aggression in patients with dementia comes from studies using topiramate. This antiepileptic drug shares some of the known mechanisms of actions of other antiepileptic drugs (e.g. sodium conductance modulation) but also modulates glutamate by decreasing alpha-amino-3-hydroxy-5-methyl-4-isoxazole-propionic acid (AMPA)-kainate receptor mediated currents (Meldrum, Epilepsia. 1996; 37 Suppi 6(4):S4-11). Fhager and colleagues (2003) (Fhager et al., International Psychogeriatrics/IPA. 2003; 15(3):307-9) conducted a retrospective evaluation of 15 severely aggressive subjects with dementia who did not respond to antipsychotic medication and then received topiramate either as monotherapy or added to an antipsychotic. Symptoms were rated using the CMAI at baseline and 2 weeks after initiating topiramate; patients in both groups showed a significant improvement in their aggressive behavior. In contrast, mibampator, a positive allosteric modulator of the glutamate AMPA receptor failed to show a benefit in a well-controlled study of Alzheimer's disease subjects with agitation/aggression (Lyketsos et al., The Journal of the Alzheimer's Association. 2011; 7(5):532-9).

Sigma-1 receptor mediated pharmacology may also play a role in dementia therapeutics and potentially in modulation of behavior. Pre-clinical studies have suggested that sigma-1 receptors are involved in many different diseases, including addiction, pain, mood disorders, psychosis, and Alzheimer's disease, among others (Su et al., Trends in Pharmacological Sciences. 2010; 31:12:557-66). Animal studies examining potential neuroprotective and behavioral effects of donepezil suggest those effects can be related to modulation of sigma-1 receptors (Maurice et al., JPET. 2006; 317(2):606-14; Villard et al., Neuropsychopharmacology. 2009; 34(6):1552-66; Marrazzo et al., NeuroReport. 2005; 16(11):1223-6). One study showed that PRE-084 or donepezil (non-selective sigma-1 agonists), when co-administered with P25-35 to mice, blocked or attenuated peptide-induced neurotoxicity. Neuroimaging studies also corroborate the potential involvement of sigma-1 receptors in Alzheimer's disease pathology. Mishina et al. (Mishina et al., Ann. Nucl. Med. 2008; 22(3):151-6) reported a lower density of sigma-1 receptors in subjects with Alzheimer's disease compared to age-matched controls in a study using positron emission tomography (PET).

Dextromethorphan

The chemistry of dextromethorphan and its analogs is described in various references such as Rodd, E. H., Ed., Chemistry of Carbon Compounds, Elsevier Publ., N.Y., 1960; Goodman and Gilman's Pharmacological Basis of Therapeutics; Choi, Brain Res. 1987; 403:333-336; and U.S. Pat. No. 4,806,543. Its chemical structure is as follows:

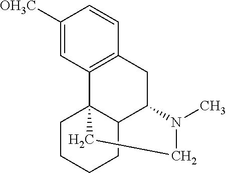

Dextromethorphan is the common name for (+)-3-methoxy-N-methylmorphinan. It is one of a class of molecules that are dextrorotatory analogs of morphine-like opioids. The term “opiate” refers to drugs that are derived from opium, such as morphine and codeine. The term “opioid” is broader. It includes opiates, as well as other drugs, natural or synthetic, which act as analgesics and sedatives in mammals.

Most of the addictive analgesic opiates, such as morphine, codeine, and heroin, are levorotatory stereoisomers (they rotate polarized light in the so-called left-handed direction). They have four molecular rings in a configuration known as a “morphinan” structure, which is depicted as follows:

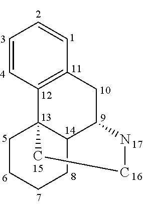 [SMILES: c1ccc2c(c1)C[C@@H]1NCC[C@]23CCCCC13]

In this depiction, the carbon atoms are conventionally numbered as shown, and the wedge-shaped bonds coupled to carbon atoms 9 and 13 indicate that those bonds rise out of the plane of the three other rings in the morphinan structure. Many analogs of this basic structure (including morphine) are pentacyclic compounds that have an additional ring formed by a bridging atom (such as oxygen) between the number 4 and 5 carbon atoms.

Many dextrorotatory analogs (which polarize light in a so-called right-handed direction) of morphine are much less addictive than the corresponding levorotatory compounds. Some of these dextrorotatory analogs, including dextromethorphan and dextrorphan, are enantiomers of the morphinan structure. In these enantiomers, the ring that extends out from carbon atoms 9 and 13 is oriented in the opposite direction from that depicted in the above structure.

Dextromethorphan has a complex pharmacology, with binding affinity to a number of different receptors, with primary activity in the central nervous system (CNS). Dextromethorphan is well known for its activity as a weak uncompetitive N-methyl-D-aspartate (NMDA) receptor antagonist (Kᵢ=1500 nM), (Tortella et al. Trends Pharmacol Sci. 1989; 10(12):501-7; Chou Y C et al., Brain Res. 1999; 821(2):516-9; Netzer R et al., Eur J Pharmacol. 1993; 238(2-3):209-16; Jaffe D B et al., Neurosci Lett. 1989; 105(1-2):227-32) with the associated potential for anti-glutamate excitatory activity. Dextromethorphan is also a potent sigma-1 agonist (Zhou G Z et al., Eur J Pharmacol. 1991; 206(4):261-9; Maurice T et al., Brain Res Brain Res Rev. 2001; 37(1-3):116-32; Cobos E J et al., Curr Neuropharmacol. 2008; 6(4):344-66), (Kᵢ=200 nM) and binds with high affinity to the serotonin transporter (SERT; Kᵢ=40 nM). Although dextromethorphan has only a moderate affinity for the norepinephrine transporter (Kᵢ=13 μM), it effectively inhibits uptake of norepinephrine (Kᵢ=240 nM) (Codd E E et al., J Pharmacol Exp Ther. 1995; 274(3):1263-70). Dextromethorphan is an antagonist of α3β4 nicotinic acetylcholine receptors, with a reported IC50 (concentration resulting in 50% inhibition) value of 0.7 μM (Damaj et al., J Pharmacol Exp Ther. 2005; 312(2):780-5).

As a result of one or more of these interactions, dextromethorphan decreases potassium-stimulated glutamate release (Annels S J et al., Brain res. 1991; 564(2):341-3), and modulates monoamine (serotonin, norepinephrine, and dopamine) neurotransmission (Codd E E et al., J Pharmacol Exp Ther. 1995; 274(3):1263-70; Maurice T et al., Pharmacol Ther. 2009; 124(2):195-206; Maurice T et al., Prog Neuropsychopharmacol Biol Psychiatry. 1997; 21(1):69-102). Dextromethorphan's antagonism of α3β34 nicotinic acetylcholine receptors (Damaj M I et al., J Pharmacol Exp Ther. 2005; 312(2):780-5) may have implications for certain CNS movement disorders and addiction (Silver A A et al., J Am Acad Child Adolesc Psychiatry. 2001; 40(9):1103-10).

Unlike some analogs of morphine, dextromethorphan has little or no agonist or antagonist activity at various other opiate receptors, including the mu (μ) and kappa (κ) classes of opiate receptors. This is highly desirable, since agonist or antagonist activity at those opiate receptors can cause undesired side effects such as respiratory depression (which interferes with breathing) and blockade of analgesia (which reduces the effectiveness of pain-killers).

Although the pharmacological profile of dextromethorphan points to clinical efficacy for several indications, when administered by itself the efficacy of dextromethorphan has been disappointing compared to placebo. Several investigators suggested that the limited benefit seen with dextromethorphan in clinical trials is associated with rapid hepatic metabolism that limits systemic drug concentrations. In one trial in patients with Huntington's disease, plasma concentrations were undetectable in some patients after dextromethorphan doses that were eight times the maximum antitussive dose (Walker et al., Clin. Neuropharmacol. 1989; 12:322-330).

Metabolism of Dextromethorphan

It has long been known that in most people (estimated to include about 90% of the general population in the United States), dextromethorphan undergoes extensive hepatic O-demethylation to dextrorphan that is catalyzed by CYP2D6 and is rapidly eliminated by the body (Ramachander et al., J. Pharm. Sci. 1977; 66(7):1047-8; and Vetticaden et al., Pharm. Res. 1989; 6(1):13-9). CYP2D6 is a member of a class of oxidative enzymes that exist in high concentrations in the liver, known as cytochrome P450 enzymes (Kronbach et al., Anal. Biochem. 1987; 162(1):24-32; and Dayer et al., Clin. Pharmacol. Ther. 1989; 45(1):34-40).

In addition to metabolizing dextromethorphan, CYP2D6 is also responsible for polymorphic debrisoquine hydroxylation in humans (Schmid et al., Clin. Pharmacol. Ther. 1985; 38:618-624). An alternate pathway is mediated primarily by CYP3A4 and N-demethylation to form 3-methoxymorphinan (Von Moltke et al., J. Pharm. Pharmacol., 1998; 50:997-1004). Both dextrorphan and 3-methoxymorphinan can be further demethylated to 3-hydroxymorphinan that is then subject to glucuronidation. The metabolic pathway that converts dextromethorphan to dextrorphan is dominant in the majority of the population and is the principle behind using dextromethorphan as a probe to phenotype individuals as CYP2D6 extensive and poor metabolizers (Kupfer et al., Lancet. 1984; 2:517-518; Guttendorf et al., Ther. Drug Monit. 1988; 10:490-498). Approximately 7% of the Caucasian population shows the poor metabolizer phenotype, while the incidence of poor metabolizer phenotype in Chinese and Black African populations is even lower (Droll et al., Pharmacogenetics. 1998; 8:325-333). A study examining the ability of dextromethorphan to increase pain threshold in extensive and poor metabolizers found antinociceptive effects of dextromethorphan were significant in poor metabolizers but not in extensive metabolizers (Desmeules et al., J. Pharmacol. Exp. Ther. 1999; 288:607-612). The results are consistent with direct effects of parent dextromethorphan rather than the dextrorphan metabolite on neuromodulation.

Rapid metabolism of dextromethorphan may be circumvented by co-administration of a CYP2D6 inhibitor along with dextromethorphan. Quinidine, a potent CYP2D6 inhibitor, has been particularly studied in this use (U.S. Pat. No. 5,206,248). The chemical structure of quinidine is as follows:

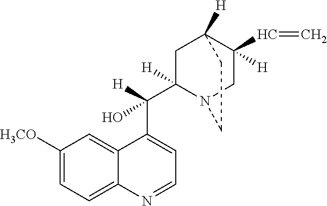

Quinidine co-administration has at least two distinct beneficial effects. First, it greatly increases the quantity of dextromethorphan circulating in the blood. In addition, it also yields more consistent and predictable dextromethorphan concentrations. Research involving dextromethorphan or co-administration of quinidine and dextromethorphan, and the effects of quinidine on blood plasma concentrations, are described in the patent literature (see, e.g., U.S. Pat. Nos. 5,166,207, 5,863,927, 5,366,980, 5,206,248, U.S. Pat. No. 5,350,756 to Smith).

While quinidine is most commonly used for coadministration, other antioxidants, such as those described in Inaba et al., Drug Metabolism and Disposition. 1985; 13:443-447, Forme-Pfister et al., Biochem. Pharmacol. 1988; 37:3829-3835, and Broly et al., Biochem. Pharmacol. 1990; 39:1045-1053, can also be co-administered with dextromethorphan to reduce its metabolism. As reported in Inaba et al., CYP2D6 inhibitors with a Ki value (Michaelis-Menton inhibition value) of 50 micromolar or lower include nortriptyline, chlorpromazine, domperidone, haloperidol, pipamperone, labetalol, metaprolol, oxprenolol, propranolol, timolol, mexiletine, quinine, diphenhydramine, ajmaline, lobeline, papaverine, and yohimbine. Compounds having particularly potent inhibitory activities include yohimbine, haloperidol, ajmaline, lobeline, and pipamperone, which have Kᵢ values ranging from 4 to 0.33 μM. In addition to the antioxidants reported above, it has also been found that fluoxetine, sold by Eli Lilly and Co. under the trade name Prozac, is effective in increasing dextromethorphan concentrations in the blood of some people. In addition, any of the following compounds may be used to inhibit CYP2D6: terbinafine, cinacalcet, buprenorphine, imipramine, bupropion, ritonavir, sertraline, duloxetine, thioridazine, metoclopramide, paroxetine, or fluvoxamine. Dosages of other antioxidants will vary with the antioxidant, and are determined on an individual basis.

Quinidine administration can convert subjects with extensive metabolizer phenotype to poor metabolizer phenotype (Inaba et al., Br. J. Clin. Pharmacol. 1986; 22: 199-200). Blood levels of dextromethorphan increase linearly with dextromethorphan dose upon co-administration with quinidine, but are undetectable in most subjects given dextromethorphan alone, even at high doses (Zhang et al., Clin. Pharmac. & Therap. 1992; 51:647-55). The observed plasma levels in rapid metabolizers following dextromethorphan co-administered with quinidine thus mimic the plasma levels observed in poor metabolizers. Accordingly, doctors should be cautious about administering quinidine to patients who may be poor metabolizers.

Neuroprotective Uses of Dextromethorphan

Dextromethorphan is widely used as a cough syrup, and it has been shown to be sufficiently safe in humans to allow its use as an over-the-counter medicine. It is well tolerated in oral dosage form, either alone or with quinidine, at up to 120 milligrams (mg) per day, and a beneficial effect may be observed when receiving a substantially smaller dose (e.g., 30 mg/day) (see, e.g., U.S. Pat. No. 5,206,248 to Smith). In addition to its use as a cough syrup, dextromethorphan has a surprisingly complex central nervous system pharmacology and related neuroactive properties that began to be elucidated and to attract the interest of neurologists in the 1980s (Tortella et al., Trends Pharmacol Sci. 1989; 10:501-7).

Neuroprotective effects of dextromethorphan were first recognized by Choi, who demonstrated that the drug attenuated glutamate-induced neurotoxicity in neocortical cell cultures (Choi. Brain Res. 1987; 403:333-6). Since this pioneering study, an increasing body of evidence has proved that dextromethorphan possesses significant neuroprotective properties in a variety of preclinical central nervous system injury models (Trube et al., Epilepsia. 1994; 35(Suppl 5):S62-7) dextromethorphan protects against seizure- and ischemia-induced brain damage, hypoxic and hypoglycemic neuronal injury, as well as traumatic brain and spinal cord injury.

Dextromethorphan's protective action in various in vitro and in vivo experiments is attributed to diverse mechanisms. Dextromethorphan has been shown to possess both anticonvulsant and neuroprotective properties, which appear functionally related to its inhibitory effects on glutamate-induced neurotoxicity (Bokesch et al., Anesthesiology. 1994; 81:470-7). Antagonism of the NMDA receptor/channel complex was originally implicated as the predominant mechanism (Trube et al., Epilepsia. 1994; 35(Suppl 5):S62-7), but dextromethorphan's action on sigma-1 receptors is also positively correlated with neuroprotective potency (DeCoster et al., Brain Res. 1995; 671:45-53). Notably, dextromethorphan's dual blockade of voltage-gated and receptor-gated calcium channels is proposed to produce a potentially additive or synergistic therapeutic benefit (Jaffe et al., Neurosci. Lett. 1989; 105:227-32; Church et al., Neurosci. Lett. 1991; 124:232-4).

Another suggested neuroprotective mechanism of dextromethorphan underlying the antagonism of p-chloroamphetamine (PCA)-induced neurotoxicity is the inhibition of serotonin (5-HT) uptake by this agent (Narita et al., Eur. J. Pharmacol. 1995; 293:277-80). It has also been proposed that dextromethorphan's interference with the inflammatory responses associated with some neurodegenerative disorders such as Parkinson's disease and Alzheimer's disease may be a novel mechanism by which dextromethorphan protects dopamine neurons in Parkinson's disease models (Liu et al., J. Pharmacol. Exp. Ther. 2003; 305:212-8; and Zhang et al., Faseb J. 2004; 18:589-91).

Abnormally elevated concentrations of glutamate are hypothesized to cause excessive excitation at the NMDA-subtype of glutamate receptors, which leads to excessive influx of sodium chloride and water, causing acute neuronal damage, and calcium, causing delayed and more permanent injury (Collins et al., Ann. Intern. Med. 1989; 110:992-1000). Considerable evidence supports roles for excitotoxicity in acute disorders such as stroke, epileptic seizures, traumatic brain and spinal cord injury, as well as in chronic, neurodegenerative disorders such as Alzheimer's disease, Parkinson's disease (PD), Huntington's disease (HD), and amyotrophic lateral sclerosis (ALS) (Mattson. Neuromolecular Med. 2003; 3:65-94). By pharmacologically inhibiting the release and subsequent deleterious actions of glutamate, dextromethorphan can serve to protect neurons in a variety of neurological disease and injury states. Dextromethorphan possesses anti-excitotoxic properties in models of NMDA and glutamate neurotoxicity (Choi et al., J. Pharmacol. Exp. Ther. 1987; 242:713-20), which are believed to be functionally related to its neuroprotective effects in models of focal and global ischemia, hypoxic injury, glucose deprivation, traumatic brain and spinal cord injury, as well as seizure paradigms (Collins et al., Ann. Intern. Med. 1989; 110:992-1000; Bokesch et al., Anesthesiology. 1994; 81:470-7; and Golding et al., Mol. Chem. Neuropathol. 1995; 24:137-50).

Dextromethorphan attenuated morphological and chemical evidence of neuronal damage in glutamate toxicity models (DeCoster et al., Brain Res. 1995; 671:45-53; and Choi et al., J. Pharmacol. Exp. Ther. 1987; 242:713-20) as well as the loss of vulnerable hippocampal (CAI) neurons in seizure (Kim et al., Neurotoxicology. 1996; 17:375-385) and global ischemia models (Bokesch et al., Anesthesiology. 1994; 81:470-7). Dextromethorphan decreased cerebral infarct size, areas of severe neocortical ischemic damage, and cortical edema after ischemia and reperfusion (Steinberg et al., Stroke. 1988a; 19:1112-1118; Ying et al., Zhongguo Yao Li Xue Bao. 1995; 16:133-6; Britton et al., Life Sci. 1997; 60:1729-40). For example, dextromethorphan decreased the incidence of frank cerebral infarction in a brain hypoxia-ischemia model (Prince et al., Neurosci. Lett. 1988; 85:291-296). In in vitro hypoxia models, dextromethorphan reduced neuronal loss and dysfunction, manifest in a decreased amplitude of the anoxic depolarization (Goldberg et al., Neurosci. Lett. 1987; 80:11-5; Luhmann et al., Neurosci. Lett. 1994; 178:171-4).

Dextromethorphan has also attenuated in vitro morphological and chemical evidence of acute glucose deprivation (Monger et al., Brain Res. 1988; 446:144-8). An effect on regional cerebral blood flow (rCBF) was suggested to contribute to the neuroprotective action of dextromethorphan in transient focal ischemia, since dextromethorphan attenuated the sharp, post-ischemic rise in rCBF during reperfusion in the ischemic core and improved delayed hypoperfusion (Steinberg et al., Neurosci. Lett. 1991; 133-225-8). A comparable attenuation of post-ischemic hypoperfusion was found with dextromethorphan in incomplete global cerebral ischemia (Tortella et al., Brain Res. 1989; 482:179-183). Furthermore, there was strong evidence of a correlated improvement in brain function, as dextromethorphan facilitated recovery of the somatosensory evoked potential (Steinberg et al., Neurosci. Lett. 1991; 133:225-8), and attenuated electroencephalographic (EEG) dysfunction in these and other ischemia studies (Ying et al., Zhongguo Yao Li Xue Bao. 1995; 16:133-6; Tortella et al., Brain Res. 1989; 482:179-183). This is consistent with findings of improved neurological function in focal ischemia (Schmid-Elsaesser et al., Exp. Brain Res. 1998; 122:121-7; and Tortella et al., J Pharmacol Exp. Ther. 1999; 291:399-408).

Similarly, the reduction in hippocampal damage in global ischemia with dextromethorphan seemed to be the basis of improvement in spatial learning and memory (Block et al., Brain Res. 1996; 741:153-9). In brain and spinal cord injury models, dextromethorphan reduced histological and biochemical damage (Duhaime et al., J. Neurotrauma. 1996; 13:79-84; Topsakal et al., Neurosurg Rev. 2002; 25:258-66), blocked traumatic spreading depression limiting the spread of traumatic injury (Church et al., J Neurotrauma. 2005; 22:277-90), and also improved the bioenergetic state (Golding et al., Mol. Chem. Neuropathol. 1995; 24:137-50).

Steinberg et al., demonstrated in a rabbit transient focal cerebral ischemia model that dextromethorphan reduced neocortical ischemic neuronal damage and edema when adequate plasma and brain levels were achieved (Steinberg et al., Neurol. Res. 1993; 15:174-80). In non-ischemic animals, dextromethorphan concentrated 7 to 30 fold in brain versus plasma, and brain levels were highly correlated with plasma levels. Plasma levels ≥500 ng/ml and brain levels ≥10,000 ng/g, or about 37 microM, were neuroprotective. While a therapeutic time window for neuroprotection has not been determined for dextromethorphan in humans, findings in preclinical ischemia models have provided some insight in this regard. Dextromethorphan was administered pre- and post-treatment in the diverse preclinical analyses. Up to 1 hour delayed treatment was found to be beneficial in models of transient focal ischemia (Steinberg et al., Neurosci. Lett. 1988; 89:193-197; and Steinberg et al., Neurol. Res. 1993; 15:174-80). This corresponds to preclinical findings for other NMDA receptor antagonists as neuroprotective drugs, which show an early window of therapeutic activity that does not exceed 1 to 2 hours (Sagratella, Pharmacol. Res. 1995; 32:1-13).

It has been demonstrated that dextromethorphan improves cerebral blood flow (CBF) in focal and global ischemia, but not in the normal brain, in such a way that it is thought to contribute to its neuroprotective action (Steinberg et al., Neurosci. Lett. 1991; 133:225-8; Tortella et al., Brain Res. 1989; 482:179-183). While the underlying mechanism(s) remain to be elucidated, an attractive suggestion has been that dextromethorphan's effect on CBF may result from blockade of VGCCs located on cerebral blood vessels resulting in vasodilation (Britton et al., Life Sci. 1997; 60:1729-40). Such an action, primarily in ischemic brain regions, could account for dextromethorphan's attenuation of post-ischemic delayed hypoperfusion (Steinberg et al., Neurosci. Lett. 1991; 133:225-8; Tortella et al., Brain Res. 1989; 482:179-183; Schmid-Elsaesser et al., Exp Brain Res. 1998; 122:121-7). However, this does not explain dextromethorphan's initial reduction of the sharp, post-ischemic rise in regional CBF in the ischemic core during reperfusion, which was observed in a focal ischemia model (Steinberg et al., Neurosci. Lett. 1991; 133:225-8). This attenuation of initial hyperemia, however, was not found by all investigators (Schmid-Elsaesser et al., Exp. Brain Res. 1998; 122:121-7). In any case, the mechanism is not known, and it is possible that the alterations in CBF seen with dextromethorphan may be secondary to its prevention of excitotoxicity with preserved autoregulation and coupling of blood flow to intact neuronal metabolism (Britton et al., Life Sci. 1997; 60:1729-40; Steinberg et al., Neurosci. Lett. 1991; 133:225-8).

Given the strong evidence for neuroprotective efficacy of dextromethorphan in preclinical in vivo models of focal and global ischemia (Bokesch et al., Anesthesiology. 1994; 81:470-7; Steinberg et al., Stroke. 1988; 19:1112-1118), as well as in vitro models of hypoxic and hypoglycemic injury (Goldberg et al., Neurosci. Lett. 1987; 80:11-5; Monyer et al., Brain Res. 1988; 446:144-8), possible clinical settings in which dextromethorphan may prove to be beneficial include ischemic stroke, cardiac arrest, and neuro- or cardiac-surgical procedures associated with a high risk of cerebral ischemia. The small clinical trial showing possible neuroprotection in perioperative brain injury in children undergoing cardiac surgery with cardiopulmonary bypass provides hope in this regard (Schmitt et al., Neuropediatrics. 1997 28:191-7). Furthermore, neuroprotective effects found in preclinical models of brain and spinal cord injury (Duhaime et al., J. Neurotrauma. 1996; 13:79-84; Topsakal et al., Neurosurg. Rev. 2002; 25:258-66), point to a possible benefit for injury caused by trauma to the central nervous system. A potential factor limiting clinical application would be the need for immediate therapy, as many experimental studies used pretreatment paradigms. However, researchers have reported promising findings of protective efficacy for dextromethorphan administered up to 1 hour after ischemic insult (Steinberg et al., Neurosci. Lett. 1988; 89:193-197; Steinberg et al., Neurol. Res. 1993; 15:174-80). Additionally, in a study of focal cerebral ischemia, 4 hours of dextromethorphan maintenance dosing was required to achieve neuroprotection (Steinberg et al., Neuroscience. 1995; 64:99-107). It has therefore been concluded that dextromethorphan shows a broader spectrum of neuroprotective activities than other NMDA receptor antagonists, which have a narrow therapeutic window (Sagratella, Pharmacol. Res. 1995; 32:1-13).

As discussed, dextromethorphan has been shown to block both NMDA receptor-operated and voltage-gated calcium channels (Jaffe et al., Neurosci. Lett. 1989; 105:227-32; Carpenter et al., Brain Res. 1988; 439:372-5), and to attenuate NMDA- and potassium-evoked increases in cytosolic free calcium concentration in neurons (Church et al., Neurosci. Lett. 1991; 124:232-4). These effects occurred at neuroprotective concentrations of dextromethorphan, and it was suggested that the drug's unique ability to inhibit calcium influx via dual routes could result in possible additive or synergistic neuroprotective effects (Jaffe et al., Neurosci. Lett. 1989; 105:227-32; Church et al., Neurosci. Lett. 1991; 124:232-4). Furthermore, presynaptic inhibition of voltage-gated calcium channels (VGCC) is suggested to underlie dextromethorphan's reduction of calcium-dependent glutamate release (Annels et al., Brain Res. 1991; 564:341-343). Calcium antagonism and inhibition of glutamate release have been implicated as potential neuroprotective mechanisms in global ischemia and hypoxic injury models (Bokesch et al., Anesthesiology. 1994; 81:470-7; Luhmann et al., Neurosci. Lett. 1994; 178:171-4; Block et al., Neuroscience. 1998; 82:791-803).

Dextromethorphan prevented the in vivo neurodegeneration of nigral dopamine neurons caused by 1-methyl-4-phenyl-1,2,3,6-tetrahydropyridine (MPTP) (Zhang et al., Faseb J. 2004; 18:589-91), and methamphetamine (METH) (Thomas et al., Brain Res. 2005; 1050:190-8) in models of Parkinson's disease via a proposed reduction in microglial activation and associated intracellular reactive oxygen species (ROS). Analogous in vitro studies showed that dextromethorphan reduced glutamate toxicity of dopamine neurons (Vaglini et al., Brain Res. 2003; 973:298-302), as well as inflammation or microglial mediated degeneration of dopamine neurons induced by lipopolysaccharide (LPS) and MPTP, even at very low concentrations of dextromethorphan (Zhang et al., Faseb J. 2004; 18:589-91; Li et al., Faseb J. 2005; 19:489-96).

Sigma-1 receptor agonist action is considered to be another important neuroprotective mechanism of dextromethorphan (Chou et al., Brain Res. 1999; 821:516-9). A sigma-1 receptor-related mechanism was implicated in kainic acid-induced seizure models (Kim et al., Life Sci. 2003; 72:769-83; Shin et al., Br. J. Pharmacol. 2005; 144:908-18), and a traumatic brain injury model (Church et al., J. Neurotrauma. 2005; 22:277-90), in which sigma-1 receptor antagonists reversed the protective effects of dextromethorphan. DeCoster et al., found a positive correlation between neuroprotective potency and sigma-1 site affinity in a glutamate toxicity model (DeCoster et al., Brain Res. 1995; 671:45-53). It must be kept in mind that the majority of sigma-1 ligands tested in this correlational study, including dextromethorphan, also have a significant to moderate affinity for the NMDA/PCP site (DeCoster et al., Brain Res. 1995; 671:45-53). However, selective sigma ligands with negligible affinity for the NMDA receptor complex also have notable in vitro neuroprotective efficacy in hypoxia/hypoglycemia models, while being less efficient against glutamate/NMDA toxicity (Maurice et al., Prog. Neuropsychopharmacol. Biol Psychiatry. 1997; 21:69-102; Maurice, Drug News Perspect. 2002; 15:617-625).

Further, selective sigma receptor agonists reduced neuronal damage in some but not other in vivo models of cerebral ischemia (Maurice et al., Prog. Neuropsychopharmacol. Biol. Psychiatry. 1997; 21:69-102). The precise role and physical nature of sigma-1 receptors in the central nervous system remains unclear. Sigma-1 sites are enriched in the plasma membrane of neuronal cells like classic proteic receptors, but they are also located on intracellular membrane organelles or dispersed throughout the cytoplasm (Maurice et al., Brain Res. Brain Res. Rev. 2001; 37:116-32). Neurosteroids and neuropeptide Y (NPY) have been proposed to be potential endogenous sigma ligands (Roman et al., Eur. J. Pharmacol. 1989; 174:301-302; Ault et al., Schizophr. Res. 1998; 31:27-36; Nuwayhid et al., J. Pharmacol. Exp. Ther. 2003; 306:934-940; Maurice et al., Jpn. J. Pharmacol. 1999; 81:125-55). Later experiments established that sigma and NPY receptor effects more likely converged at the level of signaling (Hong et al., Eur. J. Pharmacol. 2000; 408:117-125).

Sigma receptors appear to serve important neuromodulatory roles regulating the release of various neurotransmitters (Maurice et al., Brain Res. Brain Res. Rev. 2001; 37:116-32; and Werling et al., In: Matsumoto R R, Bowen W D, Su T P, eds. Sigma Receptors: Chemistry, Cell Biology and Clinical Implications. Kluwer Academic Publishers; 2006). Importantly, sigma-1 receptor agonists modulate extracellular calcium influx and intracellular calcium mobilization (Maurice et al., Brain Res. Brain Res. Rev. 2001; 37:116-32). It is hypothesized that the neuroprotective action of selective sigma ligands may relate to an indirect inhibition of ischemic-induced presynaptic glutamate release (Maurice et al., Prog. Neuropsychopharmacol. Biol. Psychiatry. 1997; 21:69-102). Therefore, the previously mentioned reduction of glutamate release by dextromethorphan (Annels et al., Brain Res. 1991; 564:341-343) could be accounted for by sigma-related inhibition of VGCC dependent synaptic release via a putative G-protein-sigma-receptor coupled mechanism, although this remains speculative (Maurice et al., Prog. Neuropsychopharmacol. Biol. Psychiatry. 1997; 21:69-102; Maurice et al., Jpn. J. Pharmacol. 1999; 81:125-55).

On the other hand, selective sigma ligands could be exerting their neuroprotective properties by acting through a putative postsynaptic and/or presynaptic intracellular target protein implicated in intracellular buffering of glutamate-induced calcium flux (Maurice et al., Brain Res. Brain Res. Rev. 2001; 37:116-32; Maurice et al., Prog. Neuropsychopharmacol. Biol. Psychiatry. 1997; 21:69-102; DeCoster et al., Brain Res. 1995; 671:45-53). An indirect modulation of NMDA receptor activity is also involved in the neuroprotective effects of certain selective sigma ligands, although the neuroprotective effects of dextromethorphan have been linked to a direct antagonism of the NMDA receptor complex (Maurice et al., Prog. Neuropsychopharmacol. Biol. Psychiatry. 1997; 21:69-102; DeCoster et al., Brain Res. 1995; 671:45-53).

Inflammatory mechanisms, such as activation of microglia, are thought to play a prominent role in the pathogenesis of Parkinson's disease (Wersinger et al., Curr. Med. Chem. 2006; 13:591-602), Alzheimer's disease (Rosenberg. Int. Rev. Psychiatry. 2005; 17:503-514), and amyotrophic lateral sclerosis (Guillemin et al., Neurodegener. Dis. 2005; 2:166-176). Studies of dextromethorphan in Parkinsonian models show that it protects dopamine neurons from inflammation-mediated degeneration in vivo and in vitro (Liu et al., J. Pharmacol. Exp. Ther. 2003; 305:212-8; Zhang et al., Faseb J. 2004; 18:589-91; and Thomas et al., Brain Res. 2005; 1050:190-8). Dextromethorphan reduced LPS- and MPTP-induced production of proinflammatory factors, including tumor necrosis factor-alpha, prostaglandin E2, nitric oxide, and especially superoxide free radicals (Liu et al., J. Pharmacol. Exp. Ther. 2003; 305:212-8; Zhang et al., Faseb J. 2004; 18:589-91; Li et al., Faseb J. 2005; 19:489-96). Specifically, dextromethorphan is proposed to act on reduced nicotinamide adenine dinucleotide phosphate (NADPH) oxidase, the primary enzymatic system in microglia for generation of ROS, since neuroprotection was not observed in NADPH oxidase-deficient animals (Liu et al., J. Pharmacol. Exp. Ther. 2003; 305:212-8; and Li et al., Faseb J. 2005; 19:489-96). Equal protection occurred at low femto- and micromolar, but not nano- and picomolar, concentrations, thus yielding a bimodal reversed W-shape dose-response relationship (Li et al., Faseb J. 2005; 19:489-96).

The investigators proposed that dextromethorphan's beneficial effects seen at low concentrations are accounted for by inhibition of microglial production of reactive oxygen species (ROS) (Zhang et al., Faseb J. 2004; 18:589-91; and Li et al., Faseb J. 2005; 19:489-96). This novel mechanism is proposed to underlie dextromethorphan's protection of dopamine neurons in both in vitro and in vivo Parkinson's disease models (Liu et al., J. Pharmacol. Exp. Ther. 2003; 305:212-8; Zhang et al., Faseb J. 2004; 18:589-91; and Thomas et al., Brain Res. 2005; 1050:190-8). There is also evidence that dextromethorphan alleviates levodopa-associated motor complications (Verhagen et al., Neurology. 1998; 51:203-206; and Verhagen et al., Mov. Disord. 1998; 13:414-417) and has helped improve Parkinsonian symptoms in some small studies (Bonuccelli et al., Lancet. 1992; 340:53; Saenz et al., Neurology. 1993; 43:15). Potential neuroprotective properties of dextromethorphan in other conditions involving neurodegenerative inflammatory processes, such as Alzheimer's disease, also appear worthy of pursuit.

Another protective mechanism of dextromethorphan implicated in a serotonergic neurotoxicity model may be its inhibition of 5-HT uptake (Narita et al., Eur. J. Pharmacol. 1995; 293:277-80). Dextromethorphan was shown to protect against the 5-HT depleting effects of PCA in two (Narita et al., Eur. J. Pharmacol. 1995; 293:277-80; Finnegan et al., Brain Res. 1991; 558:109-111) but not a third study (Farfel et al., J. Pharmacol. Exp. Ther. 1995; 272:868-75). The agent attenuated long-term reduction of 5-HT and its metabolite 5-HIAA in rat striatum and cortex. Dextromethorphan alone produced no significant changes in the concentrations of 5-HT or 5-HIAA after 10 days (Finnegan et al., Brain Res. 1991; 558:109-111).

Clinical Studies of Neuroprotection

The efficacy of dextromethorphan as a neuroprotectant was also explored in a limited number of small clinical trials in patients with amyotrophic lateral sclerosis and perioperative brain injury. Additional small studies assessed symptom improvement with dextromethorphan in Huntington's disease, Parkinson's disease, and after methotrexate (MTX) neurotoxicity. Dextromethorphan was not found to be neuroprotective in the amyotrophic lateral sclerosis trials, although the doses employed would not be expected to confer neuroprotection (Gredal et al., Acta. Neurol. Scand. 1997; 96:8-13; Blin et al., Clin. Neuropharmacol. 1996; 19:189-192; Askmark et al., J. Neurol. Neurosurg. Psychiatry. 1993; 56:197-200). A randomized, double-blind, placebo-controlled trial with amyotrophic lateral sclerosis patients (N=45) did not demonstrate an improvement in 12-month survival with a relatively low dose of dextromethorphan (150 mg/day; about 2 to 3 mg/kg) (Gredal et al., Acta. Neurol. Scand. 1997; 96:8-13). Although there was a significantly decreased rate of decline in lower extremity function scores in the dextromethorphan group, baseline differences between the groups precluded firm conclusions. A second 1-year trial (N=49) showed no significant differences in rate of disease progression between dextromethorphan- (1.5 mg/kg/day) and placebo-treated patients (Blin et al., Clin. Neuropharmacol. 1996; 19:189-192). Finally, in a third amyotrophic lateral sclerosis study (N=14) no clinical or neurophysiological parameter (relative number of axons, and compound muscle action potentials) improvements were found with dextromethorphan in a 12-week placebo-controlled, crossover study (150 mg/day), followed by an up to 6 months open trial (300 mg/day) (Askmark et al., J Neurol. Neurosurg. Psychiatry. 1993; 56:197-200). As noted above, preclinical studies have established that considerably higher doses (about 10 to 75 mg/kg, oral) are required for neuroprotective effects.

In contrast, pilot data from a small randomized, placebo-controlled study (N=13) of perioperative brain injury in children undergoing cardiac surgery with cardiopulmonary bypass suggest such an effect (Schmitt et al., Neuropediatrics. 1997; 28:191-7). Dextromethorphan (oral, high-dose 36-38 mg/kg/day, dosing started 24 hours before and ended 96 hours after surgery) reached putative therapeutic levels in plasma (maximal about 550 to 1650 ng/ml) and CSF (285 to 939 ng/ml), and significantly decreased postoperative EEG sharp waves (p=0.02). There were also reduced rates of postoperative periventricular white matter lesions (0/6 dextromethorphan vs. 2/7 placebo) and less pronounced third ventricle postoperative enlargement (diameter 0.112 cm dextromethorphan vs. 0.256 cm placebo; p=0.06), but small sample sizes may have precluded statistical significance. Adverse events were not observed. Reduced EEG sharp wave activity, ventricular enlargement, and the absence of new white matter hyperintense lesions in the dextromethorphan group may be indications of a neuroprotective effect (Schmitt et al., Neuropediatrics. 1997; 28:191-7). However, dissimilarities of treatment groups by chance precluded firm conclusions.

Symptom improvement with dextromethorphan has been observed in some, but not all studies. A retrospective chart review (N=5) evaluated dextromethorphan (oral 1-2 mg/kg) for severe sub-acute methotrexate (MTX) neurotoxicity (Drachtman et al., Pediatr. Hematol. Oncol. 2002; 19:319-327). This is a frequent complication of MTX therapy for malignant and inflammatory diseases, the multifactorial pathogenesis of which is thought to involve NMDA receptor activation (Drachtman et al., Pediatr. Hematol. Oncol. 2002; 19:319-327). Remarkably, dextromethorphan given 1 to 2 weeks after a dose of MTX completely resolved neurological symptoms, including dysarthria and hemiplegia, in all patients. It is possible that dextromethorphan could prevent permanent neurotoxic lesions associated with MTX therapy, but this was not assessed (Drachtman et al., Pediatr. Hematol. Oncol. 2002; 19:319-327).

Two small studies with Parkinson's disease patients (N=22 total) lasting a few weeks showed significant efficacy for symptom improvement at daily doses ranging between 180 and 360 mg (Bonuccelli et al., Lancet. 1992; 340:53; Saenz et al., Neurology. 1993; 43:15). A third study of Parkinson's disease patients (N=21) failed to find symptomatic improvement, but found dose-limiting side effects at 180 mg/day (Montastruc et al., Mov. Disord. 1994; 9:242-243). None of these three Parkinson's disease investigations employed neuroprotective methodology. Dextromethorphan also significantly improved levodopa-associated motor complications in two small trials (N=24 total), although with a narrow therapeutic index (Verhagen et al., Neurology. 1998; 51-203-206; and Verhagen et al., Mov. Disord. 1998; 13:414-417). Interestingly, the researchers coadministered dextromethorphan (mean dose 95 to 110 mg/day) with quinidine (100 mg BID) in these trials. These studies of levodopa-related dyskinesias and motor fluctuations, lasting a few weeks, did not specifically examine neuroprotection.

An open-label trial with Huntington's disease patients (N=11), however, found no windows of symptomatic benefit after 4 to 8 weeks of treatment, despite the achievement of a moderately high median peak tolerated dose (410 mg/day) (Walker et al., Clin. Neuropharmacol. 1989; 12:322-30). At maximum doses, performance declined on a variety of measures of Huntington's disease (functional rating scales and quantitative exam scores), consistent with dose-related side effects. Oral doses of dextromethorphan did not correlate with serum levels, which varied widely (0 to 280 ng/ml) and were randomly distributed. Nonetheless, the investigators concluded that further trials of dextromethorphan as protective therapy in Huntington's disease may be called for given the proven safety of dextromethorphan in Huntington's disease patients, its salutary effects in animal models of the disease, and the hypothesis that striatal neuronal death in Huntington's disease is mediated by NMDA receptors (Walker et al., Clin. Neuropharmacol. 1989; 12:322-30).

Several investigators suggested that the limited benefit seen with dextromethorphan in clinical trials is associated with the rapid hepatic metabolism of dextromethorphan to dextrorphan, which limits systemic drug concentrations and potential therapeutic utility (Pope et al., J. Clin. Pharmacol. 2004; 44:1132-1142; Zhang et al., Clin. Pharmacol. Ther. 1992; 51:647-55; Kimiskidis et al., Methods Find Exp. Clin. Pharmacol. 1999; 21:673-8). While difficult to extrapolate human dose requirements from animal data, it appears that dextromethorphan doses higher than typically used for antitussive effects (60 to 120 mg/day, oral), and those used in most previous neuroprotection trials, are required for neuroprotection (Gredal et al., Acta. Neurol. Scand. 1997; 96:8-13; Albers et al., Stroke. 1991; 22:1075-7; and Dematteis et al., Fundam. Clin. Pharmacol. 1998; 12:526-37). However, in the trial with Huntington's disease patients, plasma concentrations were undetectable in some patients after dextromethorphan doses that were up to 8 times the maximum antitussive dose (Walker et al., Clin. Neuropharmacol. 1989; 12:322-30).

As described above, dextromethorphan is rapidly metabolized to its primary metabolite dextrorphan. Some neuroprotective action in several preclinical models, as well as side effects, may be attributable to dextrorphan. Dextrorphan acts on many of the same sites as dextromethorphan but with different affinities or potencies. While specific reported affinities for dextromethorphan and dextrorphan at the site within the NMDA receptor-operated cation channel vary, it is generally agreed that dextrorphan has a distinctly greater affinity than dextromethorphan (Chou et al., Brain Res. 1999; 821:516-9; and Sills et al., Mol. Pharmacol. 1989; 36:160-165), and dextrorphan has been shown to be about 8 times more potent than dextromethorphan as an NMDA receptor antagonist (Trube et al., Epilepsia. 1994; 35 Suppl 5:S62-7). Dextrorphan's greater affinity at the NMDA receptor is implicated in greater neuroprotective effects of the agent compared to dextromethorphan in some models (Goldberg et al., Neurosci. Lett. 1987; 80:11-5; Monyer et al., Brain Res. 1988; 446:144-8; and Berman et al., J. Biochem. Toxicol. 1996; 11:217-26) while it is also associated with psychotomimetic disturbances (Dematteis et al., Fundam. Clin. Pharmacol. 1998; 12:526-37; Albers et al., Stroke. 1995; 26:254-258; and Szekely et al., Pharmacol. Biochem. Behay. 1991; 40:381-386).

In contrast to dextrorphan, dextromethorphan is more effective at inhibiting calcium uptake in vitro due to a 3-times more potent blockade of voltage-gated calcium flux (Jaffe et al., Neurosci. Lett. 1989; 105:227-32; Carpenter et al., Brain Res. 1988; 439:372-5; and Trube et al., Epilepsia. 1994; 35 Suppl 5:S62-7). Both drugs bind sigma-1 receptors and have been shown do so with a similar high affinity (Chou et al., Brain Res. 1999; 821:516-9; and Lemaire et al., In: Kamenka J M, Domino E F, eds. Multiple Sigma and PCP Receptor Ligands: Mechanisms for Neuromodulation and Neuroprotection? Ann Arbor, Mich.: NPP Books; 1992:287-293) or with dextromethorphan having a slightly greater (about 2 times) affinity than dextrorphan (Walker et al., Pharmacol. Rev. 1990; 42:355-402; and Taylor et al., In: Kamenka J M, Domino E F, eds. Multiple Sigma and PCP Receptor Ligands: Mechanisms for Neuromodulation and Neuroprotection? Ann Arbor, Mich.: NPP Books; 1992:767-778).

Evidence suggests that dextromethorphan binds the serotonin transporter with high-affinity (Meoni et al., Br. J. Pharmacol. 1997; 120:1255-1262), which might also confer neuroprotection in some paradigms (Narita et al., Eur. J. Pharmacol. 1995; 293:277-80), while dextrorphan does not. There may also be other sites at which dextromethorphan or dextrorphan act, and it is unclear if the parent compound and metabolite bind the exact same site within the NMDA receptor-channel complex (LePage et al., Neuropharmacology. 2005; 49:1-16). In this regard, autoradiographic studies show a differential pattern of binding for radiolabeled dextrorphan than for dextromethorphan or the other open channel blockers of the NMDA-operated cation channel, and also different from sigma sites (Roth et al., J. Pharmacol. Exp. Ther. 1996; 277:1823-1836). Such mechanistic differences could account for the differential neuroprotective efficacies of dextromethorphan and dextrorphan in various central nervous system injury models (Kim et al., Life Sci. 2003; 72:769-83; and Berman et al., J. Biochem. Toxicol. 1996; 11:217-26).

The relative neuroprotective efficacies determined in the different experiments appear to be related to differences in receptor mechanisms. Thus, dextrorphan's greater neuroprotective rank order potency compared to dextromethorphan against acute glutamate toxicity correlated with rank order for competition against [3H]MK-801 binding to the PCP site, suggesting action via the uncompetitive site within the NMDA-operated cation channel (Berman et al., J. Biochem. Toxicol. 1996; 11:217-26). On the other hand, dextromethorphan appeared to be a more potent neuroprotectant than dextrorphan in a kainic acid (KA)-induced seizure model (Kim et al., Life Sci. 2003; 72:769-83). In this paradigm, a selective sigma-1 receptor antagonist blocked dextromethorphan's neuroprotective action to a greater extent than the neuroprotective action of dextrorphan, thus implicating the sigma-1 receptor in the protective mechanism. In vitro and in vivo neuroprotection with dextromethorphan occurred in comparable concentration ranges (Choi et al., J. Pharmacol. Exp. Ther. 1987; 242:713-20; Steinberg et al., Neurol. Res. 1993; 15:174-80).

Protective effects of dextromethorphan clearly go beyond effects of dextrorphan. For instance, in a focal ischemia study, Steinberg et al., suggested that dextromethorphan's neuroprotective action was not mediated by dextrorphan, since dextrorphan plasma and brain levels were lower than neuroprotective levels of dextrorphan in the same model (Steinberg et al., Neurol. Res. 1993; 15:174-80). Furthermore, focal administration of dextromethorphan into the brain in one transient cerebral ischemia study was neuroprotective (Ying Neurol. Res. 1993; 15:174-80. Zhongguo Yao Li Xue Bao. 1995; 16:133-6). Since CYP2D6 is only expressed at low levels in the brain (Steinberg et al., Neurol. Res. 1993; 15:174-80; Tyndale. Drug Metab. Dispos. 1999; 27:924-30; Britto et al., Drug Metab. Dispos. 1992; 20:446-450), this effect and the in vitro neuroprotective properties of dextromethorphan likely do not involve metabolism to an active metabolite, at least not to the extent accomplished by first-pass, hepatic metabolism in vivo. In this regard, dextromethorphan analogs have also demonstrated protective effects against glutamate in cultured cortical neurons unrelated to the biotransformation of dextromethorphan (Tortella et al., Neurosci. Lett. 1995; 198:79-82). Another analog of dextromethorphan known not to form dextrorphan (dimemorfan) protected against seizure-induced neuronal loss with fewer PCP-like side effects (Shin et al., Br. J. Pharmacol. 2005; 144:908-18).

### Clinical Safety of Dextromethorphan

The potential safety of dextromethorphan as a neuroprotective agent has been examined in a limited number of small clinical trials. These have primarily assessed the safety/tolerability of the agent in various patient populations with both acute and chronic neurological disorders. Symptom improvement was demonstrated in some studies. Four studies were designed to evaluate neuroprotection, and two of these found neuroprotective effects (Gredal et al., Acta. Neurol. Scand. 1997; 96:8-13; and Schmitt et al., Neuropediatrics. 1997; 28:191-7). Studies with negative findings did not utilize doses sufficient for neuroprotection. The largest (N=181) dose-escalation safety and tolerance study of dextromethorphan was conducted in neurosurgery patients undergoing intracranial surgery or endovascular procedures, associated with a high risk of cerebral ischemia (Steinberg et al., J. Neurosurg. 1996; 84:860-6). Patients were given oral dextromethorphan (0.8 to 9.64 mg/kg), starting 12 hours prior to surgery and continuing up to 24 hours after surgery. Serum dextromethorphan levels correlated highly with CSF and brain levels. Dextromethorphan concentrated in brain with levels being 68-fold higher than in serum, similar to findings in animals (Steinberg et al., Neurol. Res. 1993; 15:174-80; and Wills et al., Pharm. Res. 1988; 5:PP1377). The maximum dextromethorphan levels attained were 1514 ng/ml in serum and 92,700 ng/g in brain. In 11 patients, brain and plasma levels of dextromethorphan were comparable to levels that have been shown to be neuroprotective in animal models of cerebral ischemia (serum dextromethorphan ≥500 ng/ml and brain dextromethorphan ≥10,000 ng/g). Frequent adverse events occurring at neuroprotective levels of dextromethorphan included nystagmus, nausea and vomiting, distorted vision, feeling “drunk,” ataxia, and dizziness. All symptoms, even at the highest levels, proved to be tolerable and reversible, and no patient suffered severe adverse reactions.

A few other, smaller studies have examined the role of orally administered dextromethorphan in patients with stroke (N=22 total; dextromethorphan serum levels ranging from 0 to 189 ng/ml) (Albers et al., Stroke. 1991; 22:1075-7; and Albers et al., Clin. Neuropharmacol. 1992; 15:509-14), Huntington's disease (N=11; dextromethorphan serum levels ranging from 0 to 280 ng/ml) (Walker et al., Clin. Neuropharmacol. 1989; 12:322-30), and amyotrophic lateral sclerosis (N=13; despite high doses, dextromethorphan steady-state plasma levels were detectable in only 1 of 7 patients, with a Cmax of 190 ng/ml) (Hollander et al., Ann. Neurol. 1994; 36:920-4). These studies found tolerable adverse events at a variety of doses, ranging from 120 to about 960 mg/day. Common side effects included dizziness, dysarthria, and ataxia at lower doses and hallucinations and fatigue at higher doses. The role of high-dose oral dextromethorphan in patients with amyotrophic lateral sclerosis was evaluated in a phase 1, open-label safety study (N=13) (Hollander et al., Ann. Neurol. 1994; 36:920-4). Escalating doses to a maximum tolerable dose of 4.8 to 10 mg/kg/day were given, and patients were maintained on this dose for up to 6 months. The most common adverse events were light-headedness, slurred speech, and fatigue. Side effects were usually tolerable, although they became dose-limiting in most patients. Neuropsychological testing detected no evidence of cognitive dysfunction at high doses in these amyotrophic lateral sclerosis patients (Hollander et al., Ann. Neurol. 1994; 36:920-4), which was consistent with findings in a randomized, placebo-controlled safety study of patients with a history of cerebral ischemia (N=12) (Albers et al., Clin. Neuropharmacol. 1992; 15:509-14). Overall, the safety trials demonstrate the viability of both long-term and high-dose administration of dextromethorphan to patients with conditions associated with glutamate excitotoxicity (Hollander et al., Ann. Neurol. 1994; 36:920-4). Given rapid conversion of dextromethorphan to dextrorphan, it may be that some adverse events encountered with dextromethorphan administration are actually related to dextrorphan.

The safety/tolerability of dextrorphan, the primary metabolite of dextromethorphan, was also assessed in a dose-escalation study with acute ischemic stroke patients (N=67) (Albers et al., Stroke. 1995; 26:254-258). Patients were treated with an intravenous (IV) infusion of dextrorphan within 48 hours of onset of mild-to-moderate hemispheric stroke. There was no difference in neurological outcome at 48 hours between the dextrorphan- and placebo-treated subjects, although the study was not designed to evaluate efficacy. Common transient, reversible, and generally mild to moderate adverse events included nystagmus, nausea, vomiting, somnolence, hallucinations, and agitation. Reversible hypotension was seen with higher loading doses of 200 to 260 mg/h. More severe adverse events such as apnea or deep stupor were observed in patients given the highest doses of dextrorphan. Lower doses (loading doses of 145 to 180 mg, maintenance infusions of 50 to 70 mg/h) were better tolerated and rapidly produced potentially neuroprotective plasma concentrations of dextrorphan (maximum serum levels ranging from 750 to 1000 ng/ml). Dextrorphan has been found to be almost 8 times more potent than dextromethorphan as an NMDA receptor antagonist (Trube et al., Epilepsia. 1994; 35(Suppl 5):S62-7), and to have a much greater affinity for the PCP site in the NMDA receptor complex (Chou et al., Brain Res. 1999; 821:516-9). As could be predicted, the doses tested were associated with well-defined pharmacological effects compatible with blockade of the NMDA receptor (Albers et al., Stroke. 1995; 26:254-258). These findings are consistent with animal studies in which PCP-like effects were observed with dextrorphan but not dextromethorphan (Dematteis et al., Fundam. Clin. Pharmacol. 1998; 12:526-37; and Szekely et al., Pharmacol. Biochem. Behay. 1991; 40:381-386), and in which dextromethorphan appeared to have a better therapeutic index at cerebroprotective levels (Steinberg et al., Neurol. Res. 1993; 15:174-80).

### Dosing and Bioavailability

Preclinical studies have suggested that neuroprotective effects of dextromethorphan are dependent on adequate drug concentrations in the blood reaching the brain. For example, a greater reduction in ischemic neuronal damage was observed with higher plasma levels of dextromethorphan in a rabbit model of transient focal cerebral ischemia (Steinberg et al., Neurol. Res. 1993; 15:174-80). In this study, neuroprotective brain levels were greater than 10,000 ng/g. Similarly, other studies have shown a dose-dependent decrease in ischemic or seizure-induced neuronal damage (Kim et al., Neurotoxicology. 1996; 17:375-385; Gotti et al., Brain Res. 1990; 522:290-307; and Yin et al., Zhongguo Yao Li Xue Bao. 1998; 19:223-6), although a clear relationship between dextromethorphan dose and degree of brain protection was not always found (Prince et al., Neurosci. Lett. 1988; 85:291-296; and Tortella et al., J. Pharmacol. Exp. Ther. 1999; 291:399-408). Preclinical studies in which neuroprotection was observed utilized oral dextromethorphan doses of about 10 to 75 mg/kg, whereas clinical neuroprotection studies have usually employed lower doses. As in humans, a substantial effect of first-pass metabolism on dextromethorphan bioavailability has been shown in animals, and route-specific effects on the disposition of dextromethorphan and dextrorphan in the plasma and brain must be considered (Wu et al., J. Pharmacol. Exp. Ther. 1995; 274:1431-7).

A precise relationship between dextromethorphan dose and plasma or serum concentration has not yet emerged (Walker et al., Clin. Neuropharmacol. 1989; 12:322-30; Zhang et al., Clin. Pharmacol. Ther. 1992; 51:647-55), although Steinberg et al., did observe that brain levels were 68-fold higher than serum levels in neurosurgery patients given oral dextromethorphan, and brain levels correlated highly with serum levels (Steinberg et al., J. Neurosurg. 1996; 84:860-6). (Steinberg et al., J. Neurosurg. 1996; 84:860-6). These complex pharmacokinetics are suggested to explain why even large doses of dextromethorphan (up to 960 mg/day; median 410 mg/day) produced a random distribution of, and in some cases undetectable, dextromethorphan serum concentrations (0 to 280 ng/ml) in Huntington's disease patients (Walker et al., Clin. Neuropharmacol. 1989; 12:322-30). Similarly, plasma dextromethorphan was detectable in only 1 of 7 amyotrophic lateral sclerosis patients at steady state (190 ng/ml at 3 months) despite administration of 4.8 to 10 mg/kg/day (median 7 mg/kg/day) of dextromethorphan in a safety study (Hollander et al., Ann. Neurol. 1994; 36:920-4). As described, exceptionally high dextromethorphan levels were attained by Steinberg et al., (Steinberg et al., J. Neurosurg. 1996; 84:860-6) in neurosurgery patients (maximum 1514 ng/ml in serum and maximum 9.64 mg/kg oral dose), and by Schmitt et al., (Schmitt et al., Neuropediatrics. 1997; 28:191-7) in cardiac surgery patients (maximum 1650 ng/ml in plasma and maximum 38 mg/kg/day oral dose). However, these levels were reached with high, multiple doses administered over days: neurosurgery patients were dosed beginning 12 hours before surgery and up to 24 hours after (Steinberg et al., J. Neurosurg. 1996; 84:860-6), while cardiac surgery patients were dosed starting 24 hours before until 96 hours after surgery (Schmitt et al., Neuropediatrics. 1997; 28:191-7). Such dosing regimens are not practical over the long-term, and may not be as well tolerated by patients that are awake and not under intensive care unit conditions (Schmitt et al., Neuropediatrics. 1997; 28:191-7; and Steinberg et al., J. Neurosurg. 1996; 84:860-6). Limited systemic delivery of dextromethorphan could thus, at least in part, account for disappointing trial results.

Various methods of enhancing dextromethorphan bioavailability have been proposed. For example, since the brain concentration of dextromethorphan is believed to be route dependent, parenteral administration (e.g., intravenous) has been used to avoid the first-pass effect. Similarly, the nasal route has been shown to be a viable alternative in animals, with drug absorption following intravenous profiles (Char et al., J. Pharm. Sci. 1992; 81:750-2). Nevertheless, oral administration remains the most convenient, particularly for potential treatment of chronic neurological disorders.

The most promising strategy for increasing systemically available dextromethorphan therefore appears to be the coadministration of a CYP2D6 inhibitor, such as the specific and reversible CYP2D6 inhibitor quinidine (Pope et al., J. Clin. Pharmacol. 2004; 44:1132-1142; Zhang et al., Clin. Pharmacol. Ther. 1992; 51:647-55; and Schadel et al., J. Clin. Psychopharmacol. 1995; 15:263-9). As discussed above, quinidine administration protects dextromethorphan from metabolism after oral dosing, and can convert subjects with the extensive metabolizer to the poor metabolizer phenotype. This results in elevated and prolonged dextromethorphan plasma profiles, increasing the drug's likelihood of reaching neuronal targets (Pope et al., J. Clin. Pharmacol. 2004; 44:1132-1142). This approach also improves the predictability in dextromethorphan plasma levels, as a strong linear relationship was observed between dextromethorphan dose and plasma concentration when quinidine was coadministered with increasing doses of dextromethorphan (Zhang et al., Clin. Pharmacol. Ther. 1992; 51:647-55). Finally, inhibition of dextromethorphan metabolism limits exposure to dextrorphan (Pope et al., J. Clin. Pharmacol. 2004; 44:1132-1142), which has been implicated in psychotomimetic reactions and abuse liability (Schadel et al., J. Clin. Psychopharmacol. 1995; 15:263-9).

The use of quinidine to inhibit the rapid first-pass metabolism of dextromethorphan allows the attainment of potential neuroprotective drug levels in the brain. Pope et al., demonstrated that about 30 mg quinidine is the lowest dose needed to maximally suppress O-demethylation of dextromethorphan (Pope et al., J. Clin. Pharmacol. 2004; 44:1132-1142). This dose, 30 mg twice daily (BID) given with 60 mg BID dextromethorphan, increased plasma levels of dextromethorphan 25-fold. In this manner, coadministration of 30 mg of quinidine BID with dextromethorphan in the three unsuccessful amyotrophic lateral sclerosis neuroprotection trials could have readily transformed the inadequate dextromethorphan doses into standard neuroprotective plasma concentrations. Pope et al., further showed that 120 mg daily dextromethorphan (60 mg BID) with quinidine (30 mg BID) resulted in steady state peak plasma levels of 192±45 ng/ml and an AUC0-12 of 1963±609 ng-h/ml (Pope et al., J. Clin. Pharmacol. 2004; 44:1132-1142).

A reasonable concern is that the achievement of higher dextromethorphan plasma concentrations, as well as the use of quinidine, may be associated with an increased occurrence of adverse events, particularly in patients with neurological disorders. Clinical studies to date have shown the combination of dextromethorphan and quinidine to be generally well tolerated, although the incidence of adverse events did appear to relate to dextromethorphan dose (Pope et al., J. Clin. Pharmacol. 2004; 44:1132-1142). Safety evaluations in healthy subjects (Total N=120) showed that daily doses of up to 120 mg dextromethorphan plus 120 mg quinidine administered for 1 week, resulted in mostly mild to moderate adverse events (Pope et al., J. Clin. Pharmacol. 2004; 44:1132-1142). No difference was found between the extensive and poor metabolizer phenotypes.

The most commonly reported adverse events were headache, loose stool, light-headedness, dizziness, and nausea. No electrocardiographic abnormalities were observed. In particular, there was no clinically significant change in the QTc interval. This is important, because quinidine use has been associated with QTc prolongation and the occurrence of a torsade de pointes based arrhythmia (Grace et al., Quinidine. N. Eng. J. Med. 1998; 338:35-45; and Gowda et al., Int. J. Cardiol. 2004; 96:1-6). However, the low doses of quinidine required to maximally inhibit dextromethorphan metabolism, and to reach potentially neuroprotective levels of dextromethorphan, are about 10- to 30-fold below the 600- to 1600-mg daily doses routinely used to treat cardiac arrhythmias (Grace et al., N. Eng. J. Med. 1998; 338:35-45). The mentioned studies by Pope et al., (Pope et al., J. Clin. Pharmacol. 2004; 44:1132-1142) provided the rationale for the proprietary fixed dextromethorphan/quinidine combination product AVP-923 (Zenvia™, Nuedexta®) by Avanir Pharmaceuticals (Aliso Viejo, Calif.).

Two phase 3 clinical trials testing AVP-923 for involuntary emotional expression disorder have also shown the dextromethorphan and quinidine combination to be generally well tolerated. In these trials, subjects with amyotrophic lateral sclerosis (N=140) (Brooks et al., Neurology. 2004; 63:1364-70) and multiple sclerosis (N=150) (Panitch et al., Ann. Neurol. 2006; 59:780-787) were administered daily doses of 60 mg dextromethorphan plus 60 mg quinidine BID given for 1 and 3 months resulted in mean steady state plasma levels of about 100 and 115 ng/ml, respectively. As in healthy subjects, use of AVP-923 in these patients with neurodegenerative disorders, even over a prolonged period, resulted in mostly mild to moderate adverse events. The adverse events reported more frequently with AVP-923 than its components (dextromethorphan and quinidine alone) or placebo were dizziness, nausea, and somnolence. No clinically significant changes were noted in QTc interval.

Overall, the use of low-dose quinidine to increase dextromethorphan bioavailability holds promise as a potential neuroprotective strategy. This approach allows the predictable attainment of neuroprotective levels of dextromethorphan found in preclinical studies, and the dextromethorphan/quinidine combination (e.g., the fixed combination product AVP-923) has been shown to be well tolerated in clinical trials. It was suggested over a decade ago that inhibiting the metabolism of dextromethorphan to its primary active metabolite dextrorphan is unnecessary (Hollander et al., Ann. Neurol. 1994; 36:920-4), since dextrorphan was thought to be the more potent uncompetitive NMDA receptor antagonist and protective agent (Choi et al., J. Pharmacol. Exp. Ther. 1987; 242:713-20). However, as described above, there is a continuously growing body of evidence that now demonstrates that dextromethorphan itself is neuroprotective via diverse mechanisms beyond uncompetitive NMDA receptor antagonism. In some models of central nervous system injury, dextromethorphan has a greater neuroprotective potency than dextrorphan (Kim et al., Life Sci. 2003; 72:769-83). This methodology is therefore worthy of exploration in the neuroprotective arena.

Pharmaceutical Compositions

One of the characteristics of the disclosed treatments is that the treatments function to reduce agitation and/or aggression and/or associated symptoms in subjects with dementia, such as Alzheimer's disease, without tranquilizing or otherwise significantly interfering with consciousness or alertness, and without increasing the risk of serious adverse effects. As used herein, “significant interference” refers to adverse events that would be significant either on a clinical level (they would provoke a specific concern in a doctor or psychologist) or on a personal or social level (such as by causing drowsiness sufficiently severe that it would impair someone's ability to drive an automobile). In contrast, the types of very minor side effects that can be caused by an over-the-counter drug such as a dextromethorphan-containing cough syrup when used at recommended dosages are not regarded as significant interference.

The magnitude of a therapeutic dose of dextromethorphan in combination with quinidine in the acute or chronic management of agitation and/or aggression and/or associated symptoms in subjects with dementia, such as Alzheimer's disease, can vary with the particular cause of the condition, the severity of the condition, and the route of administration. The dose and/or the dose frequency can also vary according to the age, body weight, and response of the individual patient.

In one embodiment, the dextromethorphan and quinidine are administered in a combined dose, or in separate doses administered substantially simultaneously. In one embodiment, the weight ratio of dextromethorphan to quinidine is about 1:1 or less. In some embodiments, the weight ratio is about 1:1, 1:0.95, 1:0.9, 1:0.85, 1:0.8, 1:0.75, 1:0.7, 1:0.65, 1:0.6, 1:0.55 or 1:0.5, or less. Likewise, in certain embodiments, dosages have a weight ratio of dextromethorphan to quinidine less than about 1:0.5, for example, about 1:0.45, 1:0.4, 1:0.35, 1:0.3, 1:0.25, 1:0.2, 1:0.15, 1:0.1, 1:0.09, 1:0.08, 1:0.07, 1:0.06, 1:0.05, 1:0.04, 1:0.03, 1:0.02, or 1:0.01, or less. In some embodiments, the weight ratio of dextromethorphan to quinidine is about 1:0.68, about 1:0.6, about 1:0.56, about 1:0.5, about 1:0.44, about 1:0.39, about 1:0.38, about 1:0.33, about 1:0.25, or about 1:0.22. In certain embodiments, when dextromethorphan and quinidine are administered at a weight ratio of 1:1 or less, less than 50 mg quinidine is administered at any one time. For example, in certain embodiments, quinidine is administered at about 30, 25, or 20 mg or less. In other embodiments, quinidine is administered at about 15, 10, 9.5, 9.0, 8.5, 8.0, 7.5, 7.0, 6.5, 6.0, 5.5, 5.0 mg, or less. In other embodiments, quinidine is administered at about 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg, or less. The disclosed doses can be administered amounts, therapeutic amounts, or effective amounts of dextromethorphan or quinidine.

In some embodiments, the combined dose (or separate doses simultaneously administered) at a weight ratio of 1:1 or less is administered once daily, twice daily, three times daily, four times daily, or more frequently so as to provide the patient with a certain dosage level per day, for example: 60 mg quinidine and 60 mg dextromethorphan per day provided in two doses, each dose containing 30 mg quinidine and 30 mg dextromethorphan; 50 mg quinidine and 50 mg dextromethorphan per day provided in two doses, each dose containing 25 mg quinidine and 25 mg dextromethorphan; 40 mg quinidine and 40 mg dextromethorphan per day provided in two doses, each dose containing 20 mg quinidine and 20 mg dextromethorphan; 30 mg quinidine and 30 mg dextromethorphan per day provided in two doses, each dose containing 15 mg quinidine and 15 mg dextromethorphan; or 20 mg quinidine and 20 mg dextromethorphan per day provided in two doses, each dose containing 10 mg quinidine (i.e., about 9 mg of quinidine free base) and 10 mg dextromethorphan. The total amount of dextromethorphan and quinidine in a combined dose may be adjusted, depending upon the number of doses to be administered per day, so as to provide a suitable daily total dosage to the patient, while maintaining a weight ratio of 1:1 or less.

In some embodiments, the total daily dose for dextromethorphan in combination with quinidine, for the treatment of agitation and/or aggression in subjects with Alzheimer's disease, is about 10 mg or less up to about 200 mg or more dextromethorphan in combination with about 0.05 mg or less up to about 50 mg or more quinidine. In some embodiments, a daily dose for treating agitation and/or aggression in subjects with Alzheimer's disease is about 10 mg to about 90 mg dextromethorphan in combination with about 4.75 mg to about 20 mg quinidine, in single or divided doses. In some embodiments, the total daily dose of dextromethorphan is from about 15, 16, 17, 18, 19 or 20 mg in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of mg quinidine. The disclosed doses can be administered amounts, therapeutic amounts, or effective amounts of dextromethorphan or quinidine.

In some embodiments, the daily dose for treating agitation and/or aggression in subjects with Alzheimer's disease is about 20, 21, 22, 23, 24, 25, 26, 27, 28, 29, or 30 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; or about 30, 31, 32, 33, 34, 35, 36, 37, 38, 39, or 40 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; or about 40, 41, 42, 43, 44, 45, 46, 47, 48, 49, or 50 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; or about 50, 51, 52, 53, 54, 55, 56, 57, 58, 59, or 60 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; or about 60, 61, 62, 63, 64, 65, 66, 67, 68, 69, or 70 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; or about 70, 71, 72, 73, 74, 75, 76, 77, 78, 79 or 80 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; or about 80, 81, 82, 83, 84, 85, 86, 87, 88, 89, 90 mg dextromethorphan compound in combination with about 15, 10, 9.5, 9.0, 8.5, 8.0, 7.5, 7.0, 6.5, 6.0, 5.5, 5.00, 4.95, 4.90, 4.85, 4.80, 4.75, 4.70, 4.65, 4.60, 4.55, 4.50, 4.45, 4.40, 4.35, 4.30, 4.25, 4.20, 4.15, 4.10, 4.05, 4.00, 3.95, 3.90, 3.85, 3.80, 3.75, 3.70, 3.65, 3.60, 3.55, 3.50, 3.45, 3.40, 3.35, 3.30, 3.25, 3.20, 3.15, 3.10, 3.05, 3.00, 2.95, 2.90, 2.85, 2.80, 2.75, 2.70, 2.65, 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, or 0.05 mg or less of quinidine; in single or divided doses. The disclosed doses can be administered amounts, therapeutic amounts, or effective amounts of dextromethorphan or quinidine.

In some embodiments, the daily dose of dextromethorphan and quinidine is: 45 mg dextromethorphan and 10 mg quinidine; 30 mg dextromethorphan and 10 mg quinidine; 20 mg dextromethorphan and 10 mg quinidine; 23 mg dextromethorphan and 9 mg quinidine; 15 mg dextromethorphan and 9 mg quinidine; 90 mg dextromethorphan and 20 mg quinidine; 60 mg dextromethorphan and 20 mg quinidine; 40 mg dextromethorphan and 20 mg quinidine; 46 mg dextromethorphan and 18 mg quinidine; or 30 mg dextromethorphan and 18 mg quinidine. In some embodiments, a single dose per day or divided doses (two, three, four, or more doses per day) can be administered. The disclosed doses can be administered amounts, therapeutic amounts, or effective amounts of dextromethorphan or quinidine.

In some embodiments, the therapy is initiated at a lower daily dose, for example about 15, 20, 23, 30, or 45 mg dextromethorphan in combination with about 4.75 to 10 mg quinidine per day, and increased up to about 30 or 90 mg dextromethorphan in combination with about 9.5 to 20 mg quinidine, depending on the patient's global response. In some embodiments, infants, children, patients over 65 years, and those with impaired renal or hepatic function, initially receive low doses, which may be titrated based on individual response(s) and blood level(s). Generally, a daily dosage of 15 to 90 mg dextromethorphan and 4.75 to 20 mg quinidine is well-tolerated by most patients.

As will be apparent to those skilled in the art, dosages outside of these disclosed ranges may be administered in some cases. Further, it is noted that the ordinary skilled clinician or treating physician will know how and when to interrupt, adjust, or terminate therapy in consideration of individual patient response.

Any suitable route of administration can be employed for providing the patient with an effective dosage of dextromethorphan in combination with quinidine for treating agitation and/or aggression and/or associated symptoms in subjects with dementia, such as Alzheimer's disease. For example, oral, rectal, transdermal, parenteral (subcutaneous, intramuscular, intravenous), intrathecal, topical, inhalable, and like forms of administration can be employed. Suitable dosage forms include tablets, troches, dispersions, suspensions, solutions, capsules, patches, and the like. Administration of medicaments prepared from the compounds described herein can be by any suitable method capable of introducing the compounds into the bloodstream. In some embodiments, the formulations can contain a mixture of active compounds with pharmaceutically acceptable carriers or diluents known to those of skill in the art.

The pharmaceutical compositions disclosed herein comprise dextromethorphan in combination with a CYP2D6 inhibitor, such as quinidine, or pharmaceutically acceptable salts of dextromethorphan and/or quinidine, as active ingredients and can also contain a pharmaceutically acceptable carrier, and optionally, other therapeutic ingredients.

The terms “pharmaceutically acceptable salts” or “a pharmaceutically acceptable salt thereof” refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as The Merck Index. Any suitable constituent can be selected to make a salt of an active drug discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives of dextromethorphan and/or quinidine can also be suitable for use in the compositions and methods disclosed herein. In certain embodiments, the dextromethorphan is administered in the form of dextromethorphan hydrobromide, and the quinidine is administered in the form of quinidine sulfate.

The compositions can be prepared in any desired form, for example, tables, powders, capsules, injectables, suspensions, sachets, cachets, patches, solutions, elixirs, and aerosols. Carriers such as starches, sugars, microcrystalline cellulose, diluents, granulating agents, lubricants, binders, disintegrating agents, and the like can be used in oral solid preparations. In certain embodiments, the compositions are prepared as oral solid preparations (such as powders, capsules, and tablets). In certain embodiments, the compositions are prepared as oral liquid preparations. In some embodiments, the oral solid preparations are tablets. If desired, tablets can be coated by standard aqueous or nonaqueous techniques.

In addition to the dosage forms set out above, the compounds disclosed herein can also be administered by sustained release, delayed release, or controlled release compositions and/or delivery devices, for example, such as those described in U.S. Pat. Nos. 3,845,770; 3,916,899; 3,536,809; 3,598,123; and 4,008,719.

Pharmaceutical compositions suitable for oral administration can be provided as discrete units such as capsules, cachets, sachets, patches, injectables, tablets, and aerosol sprays, each containing predetermined amounts of the active ingredients, as powder or granules, or as a solution or a suspension in an aqueous liquid, a non-aqueous liquid, an oil-in-water emulsion, or a water-in-oil liquid emulsion. Such compositions can be prepared by any of the conventional methods of pharmacy, but the majority of the methods typically include the step of bringing into association the active ingredients with a carrier which constitutes one or more ingredients. In general, the compositions are prepared by uniformly and intimately admixing the active ingredients with liquid carriers, finely divided solid carriers, or both, and then, optionally, shaping the product into the desired presentation.

For example, a tablet can be prepared by compression or molding, optionally, with one or more additional ingredients. Compressed tablets can be prepared by compressing in a suitable machine the active ingredient in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

In some embodiments, each tablet contains from about 15 mg to about 45 mg of dextromethorphan and from about 4.75 mg to about 10 mg quinidine, and each capsule contains from about 15 mg to about 45 mg of dextromethorphan and from about 15 mg to about 45 mg quinidine. In some embodiments, tablets or capsules are provided in a range of dosages to permit divided dosages to be administered. In some embodiments, the tablets, cachets or capsules can be provided that contain about 45, 30, or 20 mg dextromethorphan and about 10 mg quinidine; about 23 or 15 mg dextromethorphan and about 9 mg quinidine A dosage appropriate to the patient, the condition to be treated, and the number of doses to be administered daily can thus be conveniently selected. In some embodiments, the dextromethorphan and quinidine are incorporated into a single tablet or other dosage form. In other embodiments, the dextromethorphan and quinidine are provided in separate dosage forms.

It has been unexpectedly discovered that subjects suffering from agitation and/or aggression and/or associated symptoms in dementia, such as Alzheimer's disease, can be treated with dextromethorphan in combination with an amount of quinidine substantially lower than the minimum amount heretofore believed to be necessary to provide a significant therapeutic effect.

In some embodiments, other therapeutic agents are administered in combination with dextromethorphan. For example, dextromethorphan may be administered in combination with a compound to treat depression or anxiety.

In some embodiments, dextromethorphan and quinidine are administered as an adjuvant to known therapeutic agents for treating symptoms of Alzheimer's disease. Agents for treating symptoms of Alzheimer's disease include, but are not limited to, cholinesterase inhibitors such as donepezil, rivastigmine, galantamine and tacrine, memantine and Vitamin E.

### EXAMPLE: Agitation and Aggression in Alzheimer's Disease Clinical Study

A clinical study was conducted to determine if the combination of dextromethorphan and quinidine was effective in reducing agitation and/or aggression in subjects with Alzheimer's disease.

This investigation was a 10-week, randomized, double-dummy, placebo-controlled, multi-center study of the efficacy of oral dextromethorphan/quinidine in subjects with probable Alzheimer's disease and clinically significant agitation. The study was conducted at 42 U.S. sites, including outpatient Alzheimer's disease clinics and assisted living and nursing facilities.

Eligible participants were aged 50 to 90 years with probable Alzheimer's disease (2011 National Institute on Aging-Alzheimer Association criteria) and clinically significant agitation defined as a state of poorly organized and purposeless psychomotor activity characterized by at least one of the following: aggressive verbal (e.g., screaming, cussing); aggressive physical (e.g., destroying objects, grabbing, fighting); and nonaggressive physical (e.g., pacing, restlessness) behaviors. Eligible participants had agitation (intermittently or constantly) within 7 days prior to screening and the agitation symptoms had to be severe enough such that they interfered with daily routine and warranted pharmacological treatment. Eligible participants also scored 24 (moderately ill) on the Clinical Global Impression of Severity of Illness scale (CGIS) for agitation, and had a Mini Mental State Examination (MMSE) score of 8 to 28. Stable doses of Alzheimer's disease medications (≥2 months; memantine and/or acetylcholinesterase inhibitors), and antidepressants, antipsychotics, or hypnotics (≥1 month; including short-acting benzodiazepines and nonbenzodiazepines) were allowed; dosages were to remain stable throughout the study. Oral lorazepam (maximum 1.5 mg/day and maximum 3 days in a 7-day period) was allowed during the study as “rescue” medication for agitation if deemed necessary by the study investigator.

Exclusion criteria were non-Alzheimer's disease dementia, agitation not secondary to Alzheimer disease, hospitalization in a mental health facility, significant depression (Cornell Scale for Depression in Dementia [CSDD]≥10), schizophrenia, schizoaffective or bipolar disorder, myasthenia gravis (because quinidine use is contraindicated), or clinically significant/unstable systemic disease; history of complete heart block, corrected change in QT interval (QTc) prolongation or torsades de pointes; family history of congenital QT prolongation; history of postural or unexplained syncope within the last year; or substance/alcohol abuse within 3 years. First generation antipsychotics, tricyclic and monoamine oxidase inhibitor antidepressants were not allowed.

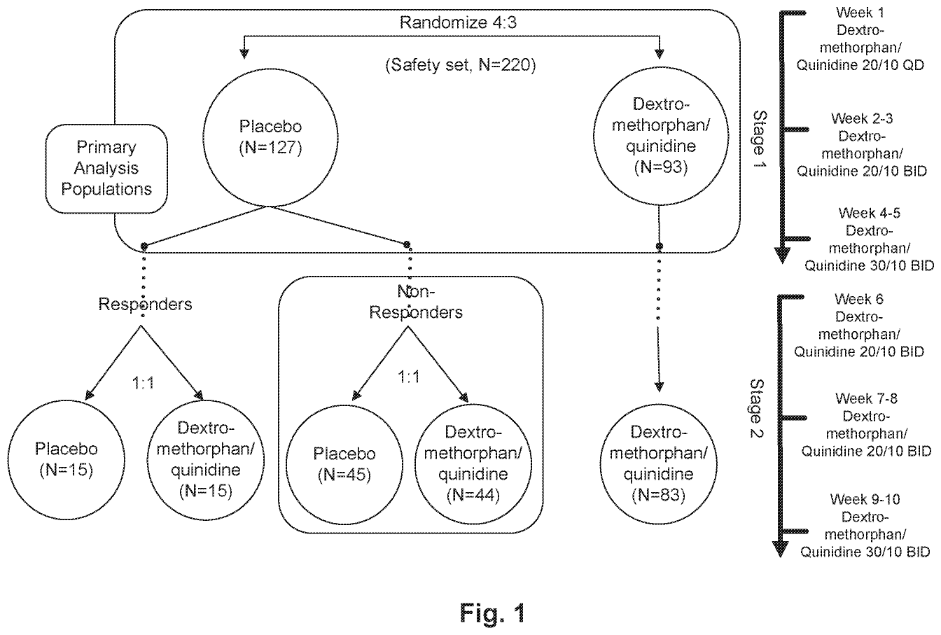

The 10-week trial had 2 consecutive double-blind 5-week stages (Stage 1 and Stage 2) (FIG. 1). Participants were randomized into Stage 1 in a 3:4 (active:placebo) ratio. Randomization in Stage 1 was stratified by baseline cognitive function (MMSE>15 vs ≥15) and agitation severity (CGIS 4-5 vs 6-7); blocked randomization ensured treatment balance in each stratum. For the initial 7 days of Stage 1 (Days 1-7), the active treatment group received AVP-923-20 (20 mg dextromethorphan and 10 mg quinidine) in the morning and placebo in the evening and the placebo group received placebo twice a day. For the following 2 weeks (Days 8-21) of Stage 1, the AVP-923 group received AVP-923-20 twice a day and the placebo group received placebo twice a day. On day 22 the dose of medication was increased for the AVP group to AVP-923-30 (30 mg dextromethorphan and 10 mg quinidine) twice a day. The AVP group continued to receive AVP-923-30 twice a day for the remaining 2 weeks of Stage 1 (Days 22-35) and participants receiving placebo continued to receive placebo twice a day.

In Stage 2, participants who received AVP-923 in Stage 1 continued to receive AVP-923 twice daily for the entire 5 week duration. Participants who received placebo in Stage 1 were stratified into two sub-groups, depending on their clinical response assessed by their Clinical Global Impression of Severity of Illness (CGIS) scores and their Neuropsychiatric Inventory (NPI) Agitation/Aggression domain scores of agitation at the end of Stage 1 (Visit 4). Participants were considered “responders” if their CGIS score for agitation was less than 3 (mildly ill) and their NPI Agitation/Aggression domain score decreased by 25% or greater from baseline. Participants who did not meet these criteria were considered “non-responders.” Each placebo sub-group (responders and non-responders) was then re-randomized in a 1:1 ratio to receive either AVP-923 or matching placebo. Participants who received placebo during Stage 1 and were re-randomized to AVP-923 in Stage 2 received AVP-923-20 in the morning and matching placebo in the evening for the initial 7 days (Stage 2, Days 36-42) of the study. Starting on Day 43, participants received AVP-923-20 twice-a-day for 2 consecutive weeks (Stage 2, Days 43-56) and starting on Day 57 participants received AVP-923-30 twice a day for the remaining 2 weeks (Stage 2, Days 57-70) until study completion.

Participants attended clinic visits at Screening, Baseline (Day 1), and on Days 8, 22, 36, 43, 57, and 70 (Visits 2-7). Including the screening phase, the length of each participant's participation in this study was approximately 14 weeks. Blood samples for measurement of drug levels in plasma were collected on Day 36 (Visit 4) and on Day 70 (Visit 7). A blood sample for cytochrome P450-2D6 (CYP2D6) genotyping was collected on Day 1 (Baseline visit).

The investigator or sponsor could discontinue a participant from the study in the event of an intercurrent illness, adverse event, other reasons concerning the health or well-being of the participant, or in the case of lack of cooperation, non-compliance, protocol violation, or other administrative reasons. In addition, participants who presented a QTc interval (Bazett-corrected QT (QTcB) or Fridericia-corrected QT (QTcF))>500 msec (unless due to ventricular pacing) or a QTc interval change from the screening electrocardiographic (ECG) result of >60 msec at any time after randomization, was withdrawn from the study. The QTc values were assessed for clinical significance and recorded. Participants who withdrew prior to study completion were asked to return to the clinic to complete the Visit 7 (End of Study) assessments. If a participant withdrew or was discontinued from the study before completion, every effort was made to document participant outcome. If the participant withdrew from the study, and consent was withdrawn by the caregiver and/or participant's representative for disclosure of future information, no further evaluations were performed, and no additional data was collected.

Participants and caregivers were instructed that the participant should take the study medication approximately every 12 hours±4 hours orally with water (morning and evening). AVP-923 and placebo were provided in identically-appearing capsules and packaged in 85 cc white plastic bottles with child-resistant caps, one bottle with white label for the morning dosing and one bottle with blue label for the evening dosing. The compositions of the AVP-923 and placebo capsules are given in Table 1.

TABLE 1

| Ingredient (amounts in mg) | AVP-923-30 | AVP-923-20 | Placebo |
|---|---|---|---|
| Dextromethorphan hydrobromide | 30.00 | 20.00 | 0 |
| USP, EP | | | |
| Quinidine sulfate dihydrate USP, EP | 10.00 | 10.00 | 0 |
| Croscarmellose sodium NF | 7.80 | 7.80 | 7.80 |
| Microcrystalline cellulose NF | 94.00 | 94.00 | 94.00 |
| Colloidal silicone dioxide NF | 0.65 | 0.65 | 0.65 |
| Lactose monohydrate NF | 116.90 | 126.90 | 156.90 |
| Magnesium stearate NE | 0.65 | 0.65 | 0.65 |

EP = European Pharmacopoeia;
USP = United States Pharmacopoeia;
NF = National Formulary

Participants and caregivers were instructed to bring any unused study medication and empty containers to the clinic on Days 8, 22, 36, 43, 57, and 70 (Visits 2-7). For this study, compliance was defined as when a participant takes at least 80% of their scheduled doses. Caregivers were provided with diary cards and were instructed to record daily the number of capsules taken and the time of administration. Diary cards were collected on Days 8, 22, 36, 43, 57, and 70 (Visits 2-7), or at the time of early study discontinuation.

### Efficacy

The primary efficacy endpoint was an improvement in the Agitation/Aggression NPI domain. Secondary efficacy endpoints included changes from baseline in NPI total score (range: 1-144), individual NPI domain scores, and NPI composite scores comprising Agitation/Aggression, Aberrant Motor Behavior, and Irritability/Lability domains plus either Anxiety (NPI4A) or Disinhibition (NPI4D). A NPI-caregiver distress score (NPI-CDS; 0-5, not at all to very severely) was captured for each positively endorsed NPI domain. Alzheimer's Disease Cooperative Study-Clinical Global Impression of Change (ADCS-CGIC; 1-7, marked improvement to marked worsening) and Patient Global Impression of Change (PGI-C), rated by a caregiver (1-7, very much improved to very much worse), scores were assessed at weeks 5 and 10 and provided measures of clinical meaningfulness. Additional secondary endpoints included ADCS-Activities of Daily Living Inventory (ADCS-ADL; 0-54, higher scores signifying better function); CSDD (0-38, higher scores signifying more severe depression); Caregiver Strain Index (CSI; 0-13, higher scores signifying higher stress levels); Quality of Life-Alzheimer Disease (QOL-AD; 13-52, with higher scores signifying better QOL); and psychotropic medication changes/rescue use of lorazepam. Cognition was assessed using the MMSE (0-30, with lower scores signifying greater cognitive impairment) and the Alzheimer Disease Assessment Scale-Cognitive Subscale (ADAS-cog; 0-70, with higher scores signifying greater cognitive impairment). Safety outcomes included adverse events (AEs), vital signs, clinical laboratory test results, and ECG results. Results for QT interval were corrected for variation in heart rate and the QTcF (QT/³√[RR]) calculations were used.

The parameters of efficacy described above were assessed at the following time points during the study: CSI and all of the NPI domains were assessed at baseline and weeks 1, 3, 5, 6, 8, and 10; ADCS-CGIC Agitation, QOL-AD (Caregiver), and ADAS-cog were assessed at baseline and weeks 5 and 10; CSDD and MMSE were assessed at screening and weeks 5 and 10; and PGI-C was assessed at weeks 5 and 10.

Primary and secondary efficacy endpoints were analyzed based on published sequential parallel comparison design (SPCD) methods (Fava et al., Psychother. Psychosom., 2003; 72(3):115-127; Chen et al., Contemp. Clin. Trials., 2011; 32(4):592-604) analyzing data from both 5-week stages with 1:1 weighting using ordinary least squares (OLS), and including all participants in stage 1 and only the rerandomized placebo nonresponders (FIG. 1) in stage 2. The primary study endpoint analysis was prespecified; no correction was performed to address multiplicity in the secondary endpoints. Dextromethorphan/quinidine and placebo groups were compared using 2-sided tests at the alpha=0.05 level of significance. Additionally, Analysis of Covariance (ANCOVA) with treatment as the fixed effect and baseline as the covariate was used to compare treatment group means at each stage and visit, separately. Finally, to simulate a 10-week parallel-arm design (as shown in FIG. 1), a pre-specified comparison of NPI Agitation/Aggression scores was conducted between participants who were randomized to receive only dextromethorphan/quinidine (n=93) or only placebo (n=66) for the entire 10 weeks of the trial (regardless of responder status). All statistical analyses were performed using SAS® version 9.1 or higher (SAS Institute, Cary, N.C., USA).

Given the use of SPCD methodology, and in order to provide assurance on findings from the primary analysis, additional exploratory sensitivity analyses of the primary endpoint were carried out. One used the repeated measures model (MMRM, prespecified) described by Doros et al (Doros et al., Stat. Med. 2013; 32(16):2767-2789) to test the potential impact of missing data and the exclusion of rerandomized placebo “responders” in stage 2. This model used all available data for the NPI Agitation/Aggression domain. Three separate models were used to estimate treatment effect and included data collected at baseline, end of stage 1, and end of stage 2, with a general model that allowed inclusion of data from intermediate visits. Based on FDA recommendation, the second sensitivity analysis of the primary endpoint using the Seemingly Unrelated Regression (SUR) method (Doros et al., Stat. Med. 2013; 32(16):2767-2789; Zellner et al., J. Am. Stat. Assoc. 1962; 57(298):348-368; Tamura and Huang, Clin. Trials. 2007; 4(4):309-317) in the SPCD, instead of the OLS method, was conducted after unblinding of the study, to address whether missing data could be missing not at random. In addition to the above, a prespecified exploratory analysis of the primary endpoint was carried out that used the same SPCD methodology described above for the primary analysis, but including both placebo responders and nonresponders who were rerandomized in stage 2.

In published treatment studies for dementia-related agitation, standard deviation (SD) estimates for change in NPI Agitation/Aggression scores range from 3.1 to 5.2 points (Herrmann et al., CNS Drugs. 2011; 25(5):425-433; Mintzer et al., Am. J. Geriatr. Psychiatry. 2007; 15(11):918-931; Herrmann et al., Dement. Geriatr. Cogn. Disord. 2007; 23(2):116-119). Assuming a SD of 5.0 points, and based on a 2-sided, 2-sample comparison of means from independent samples at the 5% significance level, a sample size of 196 participants was calculated to provide 90% power to detect a mean difference of 2.5 points. The sample size calculation was based on a parallel design as there was no precedent for an SPCD trial in treatment of agitation in subjects with Alzheimer disease.

The safety analysis set included all participants who took at least 1 dose of study medication. The modified intention-to-treat (mITT) analysis set for efficacy included all participants with a post baseline NPI Agitation/Aggression assessment in stage 1. Missing data were imputed using the last observation carried forward.

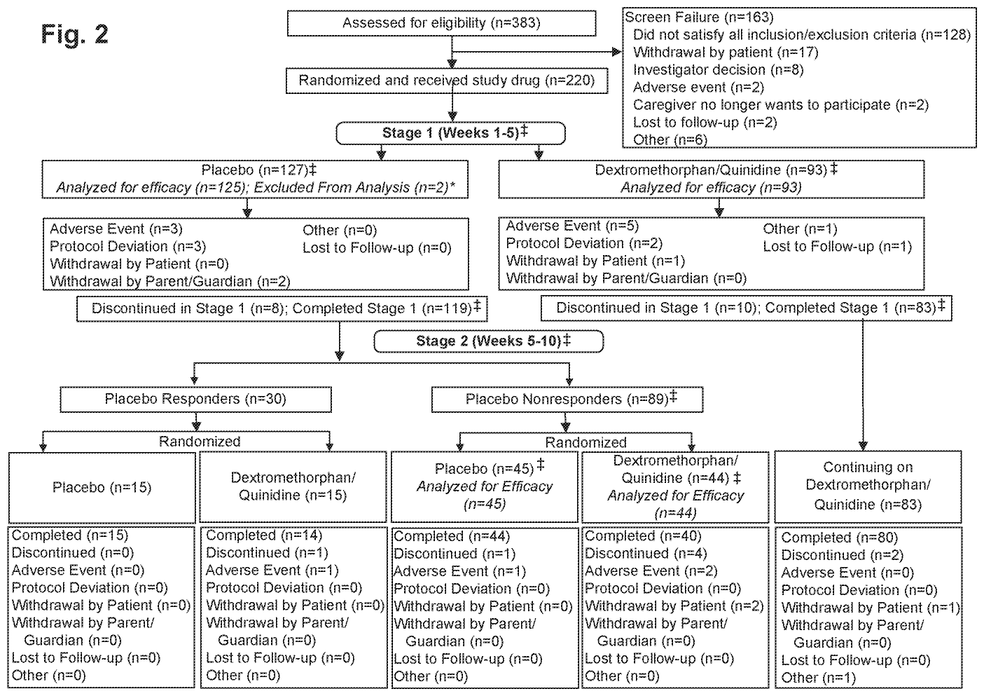

All 220 randomized participants (126 females, 94 males) were included in the safety analysis set; 218 participants composed the mITT analysis set for efficacy, and 194 (88.2%) completed the study (FIG. 2). With the SPCD and rerandomization of the placebo group upon entry into Stage 2, a total of 152 participants received dextromethorphan/quinidine (93 starting from Stage 1 and an additional 49 rerandomized from placebo in Stage 2), and 127 participants received placebo, resulting in an approximately 26.7% greater exposure for dextromethorphan/quinidine (1153 patient-weeks) than for placebo (911 patient-weeks). Seventeen (11.2%) participants discontinued while receiving dextromethorphan/quinidine and 9 (7.1%) while receiving placebo, including 8 (5.3%) and 4 (3.1%) for AEs, respectively. Participant characteristics were well-balanced across treatment groups and are provided in Table 2 and Table 3 (mITT efficacy set). The rerandomized groups in Stage 2 were also well-balanced. The mITT SPCD rerandomized placebo group characteristics are provided in Table 4.

TABLE 2

| | | | Dextromethorphan/ | |
|---|---|---|---|---|
| | Placebo | | quinidine | |
| Characteristic | (n = 127)^{a} | | (n = 93)^{a} | |
| Age (years), mean (SD) | 77.8 | (7.2) | 77.8 | (8.0) |
| Age ≥ 75 years, n (%) | 86 | (67.7) | 68 | (73.1) |
| Women, n (%) | 74 | (58.3) | 52 | (55.9) |
| Race, n (%) | | | | |
| White | 118 | (92.9) | 84 | (90.3) |
| Black or African American | 6 | (4.7) | 5 | (5.4) |
| Asian | 1 | (0.8) | 3 | (3.2) |
| Native Hawaiian or | 0 | | 1 | (1.1) |
| Other Pacific Islander | | | | |
| Other | 2 | (1.6) | 0 | |
| Ethnicity, n (%) | | | | |
| Hispanic or Latino | 13 | (10.2) | 7 | (7.5) |
| Residence, n (%) | | | | |
| Outpatient | 111 | (87.4) | 82 | (88.2) |
| Assisted living | 10 | (7.9) | 5 | (5.4) |
| Nursing home | 6 | (4.7) | 6 | (6.5) |
| Concomitant medications, n (%) | | | | |
| Acetylcholinesterase inhibitors | 95 | (74.8) | 67 | (72.0) |
| Memantine | 66 | (52.0) | 43 | (46.2) |
| Antidepressants | 65 | (51.2) | 57 | (61.3) |
| Antipsychotics | 29 | (22.8) | 16 | (17.2) |
| Benzodiazepines | 12 | (9.5) | 6 | (6.5) |
| Benzodiazepine-like derivatives | 12 | (9.5) | 6 | (6.5) |
| History of falls, n (%) | 16 | (12.6) | 16 | (17.2) |
| Rating scale scores,^{b} mean (SD) | | | | |
| CGI-S Agitation | 4.5 | (0.7) | 4.4 | (0.6) |
| NPI Agitation/Aggression | 7.0 | (2.4) | 7.1 | (2.6) |
| NPI Total | 38.0 | (18.7) | 40.1 | (19.6) |
| NPI-Aberrant Motor Behavior | 3.5 | (4.2) | 4.3 | (4.4) |
| NPI-Irritability/Lability | 5.4 | (3.2) | 5.8 | (3.7) |
| NPI 4A | 20.1 | (8.3) | 20.9 | (9.4) |
| NPI 4D | 18.5 | (9.2) | 19.8 | (9.1) |
| NPI Caregiver Distress-Agitation | 3.0 | (1.0) | 3.3 | (0.9) |
| NPI Caregiver Distress-Total | 17.0 | (8.3) | 17.9 | (8.0) |
| CSI | 6.8 | (3.6) | 6.9 | (3.2) |
| CSDD | 5.8 | (2.4) | 5.9 | (2.4) |
| QOL-AD (Patient) | 37.2 | (6.4) | 36.5 | (7.4) |
| QOL-AD (Caregiver) | 30.1 | (6.0) | 30.9 | (6.0) |
| MMSE | 17.2 | (5.8) | 17.4 | (6.0) |
| ADAS-cog | 32.0 | (15.2) | 30.6 | (14.1) |
| ADCS-ADL | 34.1 | (12.8) | 35.8 | (11.9) |
| CGIS Agitation baseline scores,^{b} n (%) | | | | |
| 4 (moderately ill) | 77 | (60.6) | 61 | (65.6) |
| 5 (markedly ill) | 40 | (31.5) | 28 | (30.1) |
| 6 or 7 (severely ill or among the most | 10 | (7.9) | 4 | (4.3) |
| extremely ill patient) | | | | |

Participant characteristics across treatment groups.
^{a}Safety analysis set at randomization;
^{b}Modified intention-to-treat analysis set for efficacy analysis (placebo, n = 125; dextromethorphan/quinidine, n = 93).

TABLE 3

| | | |
|---|---|---|
| | | Dextromethorphan/ |
| Characteristic | Placebo | quinidine |

Gender

| | | |
|---|---|---|
| n | 125 | 93 |
| Female | 74 (59.2%) | 52 (55.9%) |
| Male | 51 (40.8%) | 41 (44.1%) |

Race

| | | |
|---|---|---|
| n | 125 | 93 |
| White | 116 (92.8%) | 84 (90.3%) |
| Black or African American | 6 (4.8%) | 5 (5.4%) |
| Asian | 1 (0.8%) | 3 (3.2%) |
| American Indian or Alaska Native | 0 | 0 |
| Native Hawaiian Or Other | 0 | 1 (1.1%) |
| Pacific Islander | | |
| Other | 2 (1.6%) | 0 |

Ethnicity

| | | |
|---|---|---|
| n | 125 | 93 |
| Hispanic Or Latino | 13 (10.4%) | 7 (7.5%) |
| Not Hispanic Or Latino | 112 (89.6%) | 86 (92.5%) |

Age (years)

| | | |
|---|---|---|
| n | 125 | 93 |
| Mean | 77.6 | 77.8 |
| SD | 7.19 | 8.01 |
| Min | 56 | 53 |
| Median | 78.0 | 78.0 |
| Max | 90 | 90 |

Age Group 2 (years)

| | | |
|---|---|---|
| n | 125 | 93 |
| <75 | 41 (32.8%) | 25 (26.9%) |
| >=75 | 84 (67.2%) | 68 (73.1%) |

Patient Living Arrangements

| | | |
|---|---|---|
| n | 125 | 93 |
| Outpatient | 109 (87.2%) | 82 (88.2%) |
| Assisted Living | 10 (8.0%) | 5 (5.4%) |
| Nursing Home | 6 (4.8%) | 6 (6.5%) |

CGI-S Agitation Score

| | | |
|---|---|---|
| n | 125 | 93 |
| Mean | 4.5 | 4.4 |
| SD | 0.67 | 0.57 |
| Min | 4 | 4 |
| Median | 4.0 | 4.0 |
| Max | 7 | 6 |

CYP2D6 Metabolizer Subgroup

| | | |
|---|---|---|
| n | 121 | 85 |
| Poor metabolizers | 7 (5.8%) | 9 (10.6%) |
| Intermediate metabolizers | 48 (39.7%) | 38 (44.7%) |
| Extensive metabolizers | 65 (53.7%) | 35 (41.2%) |
| Ultra-rapid metabolizers | 1 (0.8%) | 3 (3.5%) |

Modified Intent-to-treat (mITT) efficacy population based on Stage 1 randomization.
“Extensive” metabolizers include “Normal” and “Normal or Intermediate” metabolizers.

TABLE 4

| | | |
|---|---|---|
| | | Dextromethorphan/ |
| Characteristic | Placebo | quinidine |

Gender

| | | |
|---|---|---|
| n | 45 | 44 |
| Female | 29 (64.4%) | 23 (52.3%) |
| Male | 16 (35.6%) | 21 (47.7%) |

Race

| | | |
|---|---|---|
| n | 45 | 44 |
| White | 41 (91.1%) | 42 (95.5%) |
| Black or African American | 2 (4.4%) | 2 (4.5%) |
| American Indian or Alaska Native | 0 | 0 |
| Other | 2 (4.4%) | 0 |

Ethnicity

| | | |
|---|---|---|
| n | 45 | 44 |
| Hispanic Or Latino | 6 (13.3%) | 2 (4.5%) |
| Not Hispanic Or Latino | 39 (86.7%) | 42 (95.5%) |

Age (years)

| | | |
|---|---|---|
| n | 45 | 44 |
| Mean | 77.3 | 78.3 |
| SD | 7.02 | 7.40 |
| Min | 59 | 60 |
| Median | 78.0 | 80.0 |
| Max | 89 | 90 |

Age Group 2 (years)

| | | |
|---|---|---|
| n | 45 | 44 |
| <75 | 17 (37.8%) | 13 (29.5%) |
| >=75 | 28 (62.2%) | 31 (70.5%) |

Patient Living Arrangements

| | | |
|---|---|---|
| n | 45 | 44 |
| Outpatient | 39 (86.7%) | 41 (93.2%) |
| Assisted Living | 4 (8.9%) | 2 (4.5%) |
| Nursing Home | 2 (4.4%) | 1 (2.3%) |

CGI-S Agitation Score

| | | |
|---|---|---|
| n | 45 | 44 |
| Mean | 4.6 | 4.6 |
| SD | 0.75 | 0.66 |
| Min | 4 | 4 |
| Median | 4.0 | 4.5 |
| Max | 7 | 6 |

CYP2D6 Metabolizer Subgroup

| | | |
|---|---|---|
| n | 45 | 41 |
| Poor metabolizers | 2 (4.4%) | 3 (7.3%) |
| Intermediate metabolizers | 13 (28.9%) | 19 (46.3%) |
| Extensive metabolizers | 30 (66.7%) | 18 (43.9%) |
| Ultra-rapid metabolizers | | 1 (2.4%) |

Modified Intent-to-treat (mITT) Sequential Parallel Comparison Design (SPCD) Stage 2 rerandomized placebo non-responders.
“Extensive” metabolizers include “Normal” and “Normal or Intermediate” metabolizers.

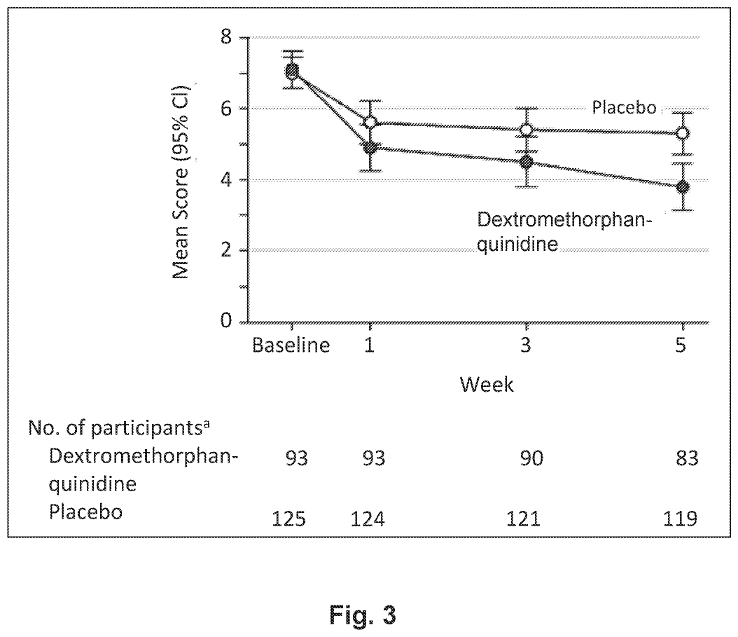

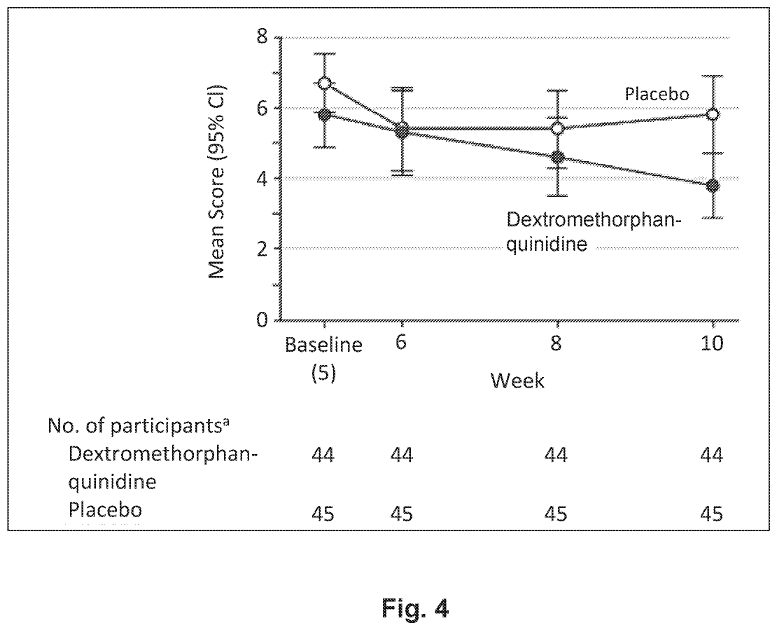

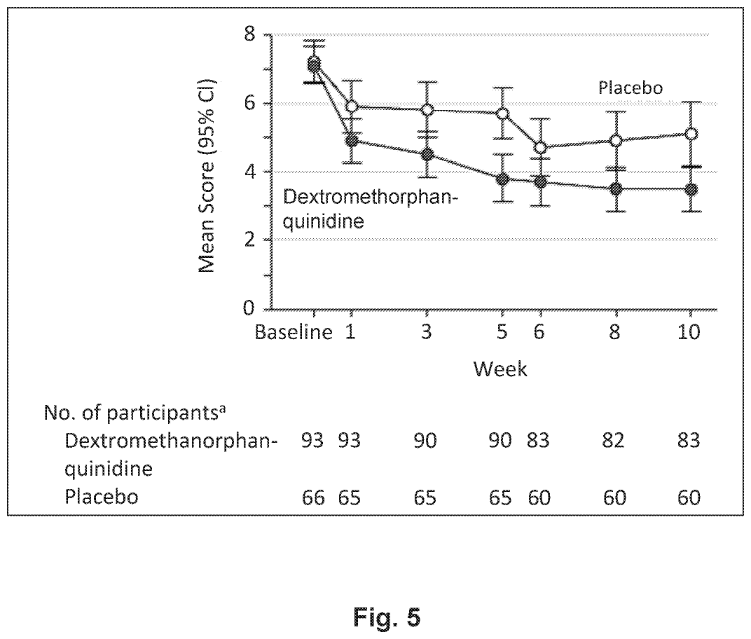

Dextromethorphan/quinidine significantly improved the NPI Agitation/Aggression score compared with placebo in the primary SPCD analysis (OLS Z-statistic: −3.95; P<0.001) in the mITT population. Results for each stage also favored dextromethorphan/quinidine over placebo (Table 5). In stage 1, mean (95% CI) NPI Agitation/Aggression scores were reduced from 7.1 (6.6, 7.6) to 3.8 (3.1, 4.5) with dextromethorphan/quinidine and from 7.0 (6.6, 7.4) to 5.3 (4.7, 5.9) with placebo (P<0.001), with a least squares (LS) mean (95% CI) treatment difference of −1.5 (−2.3, −0.7). Differential response was noted by week 1 (−0.8 [−1.5,−0.03]; P=0.04; FIG. 3). In stage 2 (placebo nonresponders rerandomized to either dextromethorphan/quinidine or placebo), mean (95% CI) NPI Agitation/Aggression scores were reduced from 5.8 (4.9, 6.7) to 3.8 (2.9, 4.7) with dextromethorphan/quinidine and from 6.7 (5.9, 7.5) to 5.8 (4.7, 6.9) with placebo (P=0.02), with an LS mean (95% CI) treatment difference of −1.6 [−2.9, −0.3]; FIG. 4). Improvement in the NPI Agitation/Aggression domain was statistically significant at week 1 and at every time point until study end, with exception of week 6 (during Stage 2). The prespecified comparison of NPI Agitation/Aggression scores between participants who were randomized to receive only dextromethorphan/quinidine (n=93) or only placebo (n=66) for the entire 10 weeks of the trial (regardless of responder status, simulating a parallel-arm design as shown in FIG. 1), also favored dextromethorphan/quinidine over placebo (LS mean treatment difference [95% Cl] of −1.8 [−2.8, −0.7]; Table 5, FIG. 5). Response to dextromethorphan/quinidine compared with placebo did not appear to differ by disease stage. The stratified randomization by baseline MMSE score (>15 vs ≤15) and baseline CGIS (4 or 5 vs. 6 or 7) resulted in balanced treatment arms for both agitation and cognitive function. Supplemental analyses conducted to assess the potential influence of these factors did not suggest a difference in response, although the sizes of some strata in these analyses were small and this observation would require confirmation in larger trials.

TABLE 5

| | | | Dextromethorphan/ | Placebo, Mean | | LS Mean | |
|---|---|---|---|---|---|---|---|
| | | N/N | quinidine, Mean | (95% CI) | P Value | Treatment | |
| | | Dextromethorphan/ | (95% CI) Change | Change from | by | Difference* | P Value |
| Parameter | Stage | quinidine/Placebo | from Baseline | Baseline | Stage^{a,b} | (95% CI) | SPCD^{h} |
| NPI-Agitation/ | 1^{a} | 93/125 | −3.3 (−3.9, −2.6) | −1.7 (−2.3, −1.2) | <.001 | −1.5 (−2.3, −0.7) | <.001 |
| Aggression^{d} | 2^{b} | 44/45 | −2.0 (−3.0, −1.0) | −0.8 (−1.9, 0.2) | .02 | −1.6 (−2.9, −0.3) | |
| | 10 wk^{c} | 93/66 | −3.6 (−4.3, −2.9) | −1.9 (−2.8, −1.0) | .001 | −1.8 (−2.8, −0.7) | N/A |
| NPI Total^{d} | 1^{a} | 93/125 | −13.5 (−17.1, −9.9) | −8.5 (−11.0, −5.9) | .03 | −4.2 (−8.0, −0.4) | .01 |
| | 2^{b} | 44/45 | −6.0 (−9.7, −2.2) | −2.5 (−6.0, 1.1) | .15 | −3.8 (−9.0, 1.4) | |
| | 10 wk^{c} | 93/66 | −16.0 (−19.5, −12.5) | −10.1 (−14.7, −5.5) | .02 | −5.7 (−10.7, −0.7) | N/A |
| NPI-Aberrant | 1^{a} | 93/125 | −1.2 (−2.0, −0.4) | −0.4 (−1.1, 0.3) | .39 | −0.4 (−1.3, 0.5) | .03 |
| Motor Behavior^{d} | 2^{b} | 44/45 | −0.8 (−1.6, −0.1) | 0.4 (−0.6, 1.3) | .04 | −1.2 (−2.4, −0.1) | |
| | 10 wk^{c} | 93/66 | −1.3 (−2.1, −0.5) | 0.1 (−0.7, 0.8) | .03 | −1.0 (−1.9, −0.1) | N/A |
| NPI-Irritability/ | 1^{a} | 93/125 | −2.2 (−3.0, −1.4) | −1.2 (−1.8, −0.6) | .09 | −0.7 (−1.5, 0.1) | 0.03 |
| Lability^{d} | 2^{b} | 44/45 | −1.0 (−2.0, 0.04) | −0.7 (−1.8, 0.5) | .14 | −0.9 (−2.2, 0,3) | |
| | 10 wk^{c} | 93/66 | −2.4 (−3.3, −1.6) | −1.8 (−2.8, −0.7) | .38 | −0.4 (−1.4, 0.6) | N/A |
| NPI4A^{d} | 1^{a} | 93/125 | −7.3 (−9.1, −5.4) | −4.5 (−6.0, −3.0) | .03 | −2.4 (−4.6, −0.2) | .001 |
| | 2^{b} | 44/45 | −4.8 (−6.9, −2.7) | −1.4 (−3.8, 1.0) | .01 | −3.9 (−7.0, −0.9) | |
| | 10 wk^{c} | 93/66 | −8.5 (−10.4, −6.7) | −5.0(−7.4, −2.5) | .01 | −3.4(−6.1, −0.7) | N/A |
| NPI 4D^{d} | 1^{a} | 93/125 | −7.6 (−9.4, −5.7) | −4.0 (−5.5, −2.6) | .006 | −3.0 (−5.1, −0.9) | <.001 |
| | 2^{b} | 44/45 | −4.6 (−6.8, −2.4) | −1.9 (−4.2, 0.4) | .02 | −3.5 (−6.5, −0.5) | |
| | 10 wk^{c} | 93/66 | −8.3 (−10.1, −6.5) | −5.0 (−7.4, −2.6) | .02 | −3.0 (−5.5, −0.4) | N/A |
| NPI Caregiver | 1^{a} | 93/125 | −1.4 (−1.6, −1.0) | −0.6 (−0.8, −0.4) | <.001 | −0.7 (−1.0, −0.3) | .01 |
| Distress- | 2^{b} | 44/45 | −0.5 (−0.9, −0.004) | −0.7 (−1.2, −0.2) | .49 | −0.2 (−0.8, 0.4) | |
| Agitation^{d} | 10 wk^{c} | 93/66 | N/A | N/A | N/A | N/A | N/A |
| NPI Caregiver | 1^{a} | 93/125 | −6.6 (−8.2, −5.0) | −3.6 (−4.8, −2.5) | N/A | N/A | .01 |
| Distress-Total^{d} | 2^{b} | 44/45 | −2.6 (−4.3, −1.0) | −2.0 (−3.8, −0.3) | N/A | N/A | |
| | 10 wk^{c} | 93/66 | N/A | N/A | N/A | N/A | N/A |
| CSI^{d} | 1^{a} | 93/125 | −1.2 (−1.7, −0.7) | −0.6 (−0.9, −0.2) | .03 | −0.6 (−1.2, −0.1) | .05 |
| | 2^{b} | 44/45 | −0.2 (−0.7, 0.3) | 0.1 (−0.5, 0.6) | .42 | −0.3 (−1.0, 0.4) | |
| | 10 wk^{c} | 93/66 | −1.2 (−1.7, 0.6) | −0.4 (−0.9, 1.3) | .04 | −0.8 (−1.6, −0.02) | N/A |
| CSDD [symbol] | 1^{a} | 88/123 | −1.0 (−1.8, −0.3) | 0.6(−0.1, 1.3) | .002 | −1.6 (−2.5, −0.6) | .02 |
| | 2^{b} | 43/44 | −0.9 (−1.8, −0.004) | −0.7 (−1.5, 0.1) | .75 | −0.2 (−1.3, 0.9) | |
| | 10 wk^{c} | 88/64 | −1.2 (−2.0, −0.4) | 0.4 (−0.6, 1.5) | .03 | −1.3 (−2.6, −0.1) | N/A |
| ADCS-CGIC | 1^{a} | 88/123 | 3.0 (2.8, 3.3) | 3.6 (3.4, 3.8) | <.001 | −0.6 (−0.9, −0.3) | <.001 |
| Agitation^{e} | 2^{b} | 42/42 | 3.3 (2.9, 3.6) | 3.7 (3.3, 4.2) | .07 | −0.5 (−1.0, 0.1) | |
| | 10 wk^{c} | 82/59 | 2.7 (2.3, 3,1) | 3.3 (3.0, 3,7) | .02 | −0.5 (−0.9, −0.1) | N/A |
| PGI-C^{g} | 1^{a} | 88/123 | 3.1 (2.8, 3.3) | 3.6 (3.4, 3.8) | .001 | −0.6 (−0.9, −0.2) | .001 |
| | 2^{b} | 43/44 | 3.2 (2.8, 3.6) | 3.8 (3.3, 4.2) | .04 | −0.6 (−1,1, −0.1) | |
| | 10 wk^{c} | 81/59 | 2.9 (2.7, 3.2) | 3.5 (3.2, 3.8) | .007 | −0.6 (−1.0, −0.2) | N/A |
| QOL-AD | 1^{a} | 87/116 | 1.3 (−0.03, 2.6) | 0.0 (−1.0, 0.9) | .14 | 1.1 (−0.4, 2.6) | .16 |
| (Patient)^{e} | 2^{b} | 40/40 | 1.5 (−0.1, 3.1) | 0.7 (−0.7, 2.0) | .50 | 0.7 (−1.4, 2.7) | |
| | 10 wk^{c} | 87/61 | 0.7 (−0.7, 2.1) | 0.5 (−1.1, 2.0) | .96 | −0.1 (−2.0, 1.9) | N/A |
| QOL-AD | 1^{a} | 88/123 | 0.4 (−0.5, 1.3) | 0.3 (−0.5, 1.1) | .63 | 0.3 (−0.9, 1.5) | .47 |
| (Caregiver)^{e,i} | 2^{b} | 43/43 | −0.3 (−1.5, 0.9) | 0.9 (−0.4, 2.2) | .24 | 1.1 (−2.8, 0.7) | |
| | 10 wk^{c} | 88/64 | 1.3 (0.2, 2.4) | 0.9 (−0.5, 2.4) | .28 | 0.9 (−0.7, 2.6) | N/A |
| ADCS-ADL^{e} | 1^{a} | 88/123 | −0.9 (−1.8, −0.04) | −0.8 (−1.5, −0.1) | .90 | −0.1 (−1.2, 1.1) | .16 |
| | 2^{b} | 43/44 | −2.0 (−3.4, −0.5) | −0.6 (−1.7, 0.4) | .12 | −1.4 (−3.1, 0.4) | |
| | 10 wk^{c} | 88/64 | −0.8 (−1.8, 0.2) | −1.8 (−2.9, 0.7) | .17 | 1.0 (−0.5, 2.5) | N/A |
| MMSE Total | 1^{a} | 88/122 | 0.2 (−0.4, 0.9) | −0.3(−0.8, 0.2) | .20 | 0.5(−0.3. 1,3) | .05 |
| Score [symbol] | 2^{b} | 42/44 | 0.3 (−0.5, 1.2) | −0.5 (−1.3, 0.2) | .15 | 0.8 (−0.3, 2.0) | |
| | 10 wk^{c} | 88/63 | 0.1 (−0.5, 0.8) | −0.6 (−1.5, 0.3) | .21 | 0.7 (−0.4, 1.8) | N/A |
| ADAS-cog^{e} | 1^{a} | 87/121 | −0.9 (−2.5, 0.6) | 0.3 (−5.7, 1.3) | .11 | −1.4 (−3.0, 0.3) | .20 |
| | 2^{b} | 42/43 | 0.3 (−1.4, 1.9) | 0.8 (−0.7, 2.3) | .64 | −0.5 (−2.8, 1,7) | |
| | 10 wk^{c} | 81/58 | −0.7 (−1.9, 0.7) | 1.2 (−0.2, 2.4) | .07 | −1.7 (−3.5, 0.2) | N/A |

*Treatment difference: dextromethorphan/quinidine-placebo;
^{a}Stage 1: Includes all participants and measures change from stage 1 baseline to week 5 for each outcome;
^{b}Staae 2: Includes only rerandomized placebo nonresponders from stage 1 and measures change from stage 2 baseline (weeks) to week 10 for all outcomes except PGI-C (original stage 1 baseline to week 10);
^{c}The 10-week analysis includes only participants who remained on their original treatment for their entire study participation (i.e., took only dextromethorphan/quinidine or only placebo, thereby simulating a parallel comparison design), and measures stage 1 baseline to week 10;
^{d}Assessed at baseline, weeks 1, 3, 5, 6, 8, and 10;
^{e}Assessed at baseline, weeks 5 and 10; [symbol]
Assessed at screening, weeks 5 and 10;
^{g}Assessed at weeks 5 and 10.
^{h}SPCD (sequential parallel comparison design) analysis was protocol-specified for the primary efficacy analysis and combines results from all patients in Stage 1 and from ″placebo nonresponders″ re-randomized in Stage 2, based on a 50/50 weighting of the NPI agitation/aggression domain for each stage of the study; [symbol]
For the QOL-AD (caregiver), the caregiver rates the patient's quality of life;
P value by Stage based on Analysis of Covariance (ANCOVA) analysis;
P value for SPCD analysis based on Ordinary Least Squares (OLS). [symbol]
indicates data missing or illegible when filed

SPCD analysis of prespecified secondary outcomes (Table 5) showed significant improvement favoring dextromethorphan/quinidine on global rating scores (PGI-C and CGIC), NPI total, NPI Aberrant Motor Behavior and Irritability/Lability domains, NPI 4A and 4D composites, NPI caregiver distress (both Agitation/Aggression domain and total), CSI, and CSDD. Results for changes in QOL-AD, ADCS-ADL, MMSE, and ADAS-cog (an exploratory outcome) were not significant vs placebo. Post hoc analyses showed similar improvement in NPI Agitation/Aggression scores with dextromethorphan/quinidine in participants taking concomitant acetylcholinesterase inhibitors, memantine, antidepressants, or antipsychotics compared with those not receiving these agents. Lorazepam rescue was used by 10 of 152 (6.6%) and 13 of 125 (10.4%) participants while receiving dextromethorphan/quinidine and placebo, respectively. At the end of the 10-week treatment, 45.1% of dextromethorphan/quinidine-only treated participants (n=82) were judged to be “much improved” or “very much improved” on ADCS-CGIC vs 27.1% of participants who took only placebo (n=59).

### Safety and Tolerability

Dextromethorphan/quinidine was generally well tolerated in this population receiving multiple concomitant medications and was not associated with cognitive impairment. Treatment-emergent adverse events (TEAEs) were attributed based on treatment assignment at the time of occurrence. TEAEs were reported by 93 of 152 (61.2%) and 55 of 127 (43.3%) participants (safety set) during treatment with dextromethorphan/quinidine or placebo, respectively. The most commonly occurring TEAEs (>3%) were fall (8.6% vs 3.9%), diarrhea (5.9% vs 3.1%), urinary tract infection (5.3% vs 3.9%), dizziness (4.6% vs 2.4%) and agitation (3.3% vs 4.7%) for dextromethorphan/quinidine vs placebo, respectively. Serious adverse events (SAEs) occurred in 12 (7.9%) of participants receiving dextromethorphan/quinidine and in 6 (4.7%) receiving placebo. SAEs in participants receiving dextromethorphan/quinidine included chest pain (n=2), anemia, acute myocardial infarction (occurring 2 days after dosing ended), bradycardia, kidney infection, femur fracture, dehydration, colon cancer, cerebrovascular accident, aggression, and hematuria (n=1 each). SAEs in participants receiving placebo included idiopathic thrombocytopenic purpura, vertigo, pneumonia, gastroenteritis, contusion, transient ischemic attack, and agitation (n=1 each). Eight (5.3%) participants receiving dextromethorphan/quinidine and 4 (3.1%) receiving placebo discontinued treatment owing to AEs, including 4 (2.6%) and 2 (1.6%), respectively, for SAEs. No deaths occurred during the study.

Of the 13 participants who fell while receiving dextromethorphan/quinidine, 9 had a prior history of falls. Three fell 2 to 4 days after study completion, and 1 participant fell twice within 24 hours of receiving lorazepam rescue in both instances; no participants who fell while receiving placebo had a history of falls. Two falls were associated with serious AEs (SAEs): femur fracture on dextromethorphan/quinidine and contusion on placebo.

No clinically meaningful between-group differences in ECG parameters were observed. The mean (SD) QTcF was 5.3 (14.06) and −0.3 (12.96) msec for participants receiving dextromethorphan/quinidine (n=138) and placebo (n=60), respectively, at final visit. Fifteen (10.3%) receiving AVP 923 and 8 (6.7%) receiving placebo had a QTcF change 230 msec at any visit; one participant on placebo had a QTcF change >60 msec. No participant had a QTcF>500 msec.

It is clear from the data presented in Table 5 and FIG. 3, FIG. 4, and FIG. 5, that the combination of dextromethorphan and quinidine is significantly effective in treating agitation and aggression in patients with probable Alzheimer's disease compared to placebo. Additionally, this combination was generally well tolerated in this elderly population and was not associated with cognitive impairment, sedation, or clinically significant QTc prolongation.

The above description discloses several methods and materials of the present invention. This disclosure is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the disclosure.

1-22. (canceled)

23. A method of treating agitation in a subject with dementia comprising administering to the subject dextromethorphan in combination with quinidine, wherein the amount of dextromethorphan administered is 30 mg/day and the amount of quinidine administered is 15 mg/day.
